(19) United States
(12) Patent Application Publication
(10) Pub. No.: US 2024/0400583 A1
(43) Pub. Date: Dec. 5, 2024

(54) TRICYCLIC SULFAMIDES AND SULTAMS

(71) Applicant: Merck Sharp & Dohme LLC, Rahway, NJ (US)

(72) Inventors: John P. Caldwell, Ringwood, NJ (US); Ping Lan, Plainsboro, NJ (US); Hongwu Wang, Westfield, NJ (US); Paul Wehn, Vernon Hills, IL (US)

(73) Assignee: Merck Sharp & Dohme LLC, Rahway, NJ (US)

(21) Appl. No.: 18/698,401

(22) PCT Filed: Oct. 7, 2022
(86) PCT No.: PCT/US2022/045960

§ 371 Date: Apr. 4, 2024

Related U.S. Application Data
(60) Provisional application No. 63/254,578, filed on Oct. 12, 2021.

(51) Int. Cl. C07D 513/04; A61K 31/549

(52) U.S. Cl. CPC C07D 513/04; A61K 31/549

(57) ABSTRACT

Compounds of formula I, that modulate DCAF15 and induce RBM139 protein degradation, pharmaceutical compositions containing these compounds, and methods of using these compounds for treating diseases associated with RBM39 protein activity and DCAF15 modulation are described herein.

### BACKGROUND OF THE INVENTION

The frequent observation of aberrant pre-mRNA splicing in many cancers may offer opportunities for the development of novel therapeutic agents. For example, RBM39 protein is associated with core components of the spliceosome. Loss or reduction of amount of RBM39 protein can alter the frequency of alternative splicing events resulting in exon skipping and intron retention. Such events may trigger selective lethality in cancer cells reliant on altered splicing or induce expression of splicing-derived neoantigens that can be exploited for therapy. For example, it was found that anticancer sulfonamide, indisulam, targets pre-RNA splicing by inducing RBM39 degradation via recruitment of DCAF15 (See T. Han, et al., Anticancer sulfonamides target splicing by inducing RBM39 degradation via recruitment to DCAF15″, Science 356, eeal. 3755, (2017).

RBM39 protein is required for acute myeloid leukemia (AML) maintenance through misspicing of HOXA9 target genes and it has been found that RBM39 loss alters splicing of mRNAs essential for AML cell growth (See E. Wang et al., “Targeting an RNA-Binding Protein network in Acute Myeliod Leukemia”, Cancer Cell 35, 369-382, (2019) and D. Hsiehchen, et al., “Biomarkers for RBM39 degradation in acute myeloid leukemia”, Springer Nature, February 2020)). It is also believed that compounds able to degrade RBM39 may be effective in treating cancers such as colon, EZH2 mutant limphoma, and melanoma.

### SUMMARY OF THE INVENTION

The present invention relates to novel compounds, to pharmaceutical compositions comprising the compounds, to a process for making the compounds and to the use of the compounds in therapy. More particularly, it relates to certain tricyclic sulfamide and sultam derivatives useful in potential disease treatment via proteasomal degradation mechanism by modulating E3 ubiquitin ligase DCAF15 and recruiting neosubstrates such as RBM39 for degradation.

The invention identifies a class of tricyclic sulfamide and sultam derivatives which are capable of mediating the selective degradation of RBM39, an RNA binding protein associated with core components of the spliceosome. Loss or diminished amounts of RBM39 protein can alter the frequency of alternative splicing events resulting in exon skipping and intron retention.

Such events may trigger selective lethality in cancer cells reliant on altered splicing or induce expression of splicing-derived neoantigens that can be exploited for therapy. In addition to RBM39 degradation, the disclosed compounds have superior binding affinities to the E3 ligase DCAF15. Small molecule binding to DCAF15 enables proximilization of RBM39 to the cullin ring ligase system for sequential ubiquitylation and proteasomal degradation of RBM39. It may be possible to harness the improved affinity of the disclosed compounds towards DCAF15 for novel applications such as in bifunctional degradation or in the construction of libraries to prospect for molecular glues capable of mediating the degradation of neo-substrates.

In one aspect, the present invention provides a compound of Formula I:

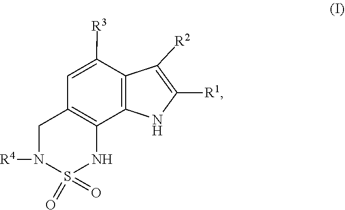

or a pharmaceutically acceptable salt thereof.

In another aspect, the present disclosure provides a pharmaceutical composition comprising a compound of Formula I or a pharmaceutically acceptable salt thereof, and a pharmaceutically acceptable diluent or carrier.

In another aspect, the present disclosure provides a method of inducing degradation of RBM39 protein, comprising a complex formation between RBM39 protein, an effective amount of compound described herein, such as compound of Formula I, and E3 ligase DCAF15. The complex formation may take place in vivo or in vitro.

In another aspect, the present disclosure provides a method of treating cancer in a subject in need thereof, comprising administering to the subject an effective amount of a compound described herein, such as a compound of Formula I, or a pharmaceutically acceptable salt thereof.

In another aspect, the present invention provides a compound, such as a compound of Formula I, or a pharmaceutically acceptable salt thereof, for use as a medicament for the treatment of cancer.

### DETAILED DESCRIPTION OF THE INVENTION

The present invention includes compounds of formula I

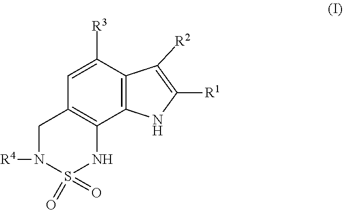

or a pharmaceutically acceptable salt thereof, wherein:
R¹ is selected from H, (C₁-C₆)alkyl, aminocarbonyl(C₁-C₆)alkyl, (C₁-C₆)alkylaminocarbonyl(C₀-C₆)alkyl, carboxy (C₁-C₆)alkyl, hydroxycarbonyl(C₀-C₆)alkyl), and (C₁-C₆)alkoxycarbonyl(C₀-C₆)alkyl);
R² is H, (C₁-C₆)alkyl, cyano, —(C₁-C₆) OH, halogen, or (C₁-C₆)haloalkyl;
R³ is H, (C₁-C₆)alkyl, cyano, —(C₁-C₆) OH, halogen, or (C₁-C₆)haloalkyl;
R⁴ is selected from (C₁-C₆)alkoxy ((C₁-C₆)alkyl), heterocycloalkyl((C₀-C₆)alkyl), heteroaryl((C₀-C₆)alkyl), —C(═O) (C₁-C₆)alkyl-(O—(C₁-C₆)alkyl)ₙ, and —(C₁-C₆)alkyl-(O—(C₁-C₆))ₙ, wherein R⁴ is substituted with 0, 1, 2, or 3 R⁵ substituents;
each n is independently 1, 2, 3, or 4;
each R⁵ independently is selected from cyano, (C₁-C₆)haloalkyl, halogen, hydroxy, oxo, (C₁-C₆)alkyl, (C₁-C₆)alkoxy ((C₀-C₆)alkyl), —SO₂ (C₁-C₆)alkyl, amino, —C(═O) (C₁-C₆)alkyl-(O—(C₁-C₆)alkyl)ₘ, —(C₁-C₆)alkyl-(O—(C₁-C₆)alkyl)ₘ, amino (C₁-C₆)akylcarbonyl, (C₁-C₆)alkylcarboxy, (C₁-C₆)alkylcarbonyl, and —(C₁-C₆)alkylOH, wherein each R⁵ independently is substituted with 0, 1, 2, or 3 R⁶ substituents;
each R⁶ is independently selected from amino, (C₁-C₆)alkylcarboxy, (C₁-C₄)alkyl, halogen and hydroxy;
each m independently is 1, 2, or 3; and provided that when R⁴ is (C₁-C₆)alkoxy ((C₁-C₆)alkyl), heterocycloalkyl((C₀-C₆)alkyl), or heteroaryl((C₀-C₆)alkyl) then R¹ is other than hydrogen.

In a first embodiment of the invention, R¹ is selected from hydrogen, aminocarbonyl(C₁-C₆)alkyl, (C₁-C₆)alkylaminocarbonyl(C₀-C₆)alkyl, carboxy (C₁-C₆)alkyl, hydroxycarbonyl(C₀-C₆)alkyl), and (C₁-C₆)alkoxycarbonyl(C₀-C₆)alkyl), and the other groups are as provided in the general formula above.

In a second embodiment of the invention, R¹ is selected from hydrogen, aminocarbonylmethyl, aminocarbonylethyl, methylaminocarbonyl, ethylaminocarbonyl, propylaminocarbonyl, methylcarboxy, ethylcarboxy, carboxypropyl, carboxyisopropyl carboxy butyl, and aminocarbonylpropyl, aminocarbonylethyl, and aminocarbonylmethyl and the other groups are as provided in the general formula above.

In a third embodiment of the invention R¹ is selected from hydrogen, methylaminocarbonyl, methylcarboxy, ethylcarboxy, carboxypropyl, and aminocarbonylpropyl and the other groups are as provided in the general formula above.

In a fourth embodiment of the invention, R² is hydrogen, halogen, or (C₁-C₆)haloalkyl and the other groups are as provided in the general formula above, or as in the first through third embodiments.

In a fifth embodiment of the invention, R² is hydrogen, chloro, fluoro or bromo and the other groups are as provided in the general formula above, or as in the first through third embodiments.

In a sixth embodiment of the invention R² is chloro, and the other groups are as provided in the general formula above, or as in the first through third embodiments.

In a seventh embodiment of the invention, R³ is is hydrogen, halogen, or (C₁-C₆)haloalkyl and the other groups are as provided in the general formula above, or as in the first through sixth embodiments.

In an eighth embodiment of the invention, R³ is hydrogen, chloro, fluoro or bromo and the other groups are as provided in the general formula above, or as in the first through sixth embodiments.

In a ninth embodiment of the invention R³ is chloro, and the other groups are as provided in the general formula above, or as in the first through sixth embodiments.

In a tenth embodiment of the invention, R⁴ is selected from (C₁-C₄)alkoxy ((C₁-C₄)alkyl), heterocycloalkyl((C₀-C₄)alkyl), heteroaryl((C₀-C₄)alkyl), —C(═O) (C₁-C₆)alkyl-(O—(C₁-C₆)alkyl)ₙ, and —(C₁-C₆)alkyl-(O—(C₁-C₆alkyl))ₙ, wherein R⁴ is substituted with 0, 1, 2, or 3 R⁵ substituents and the other groups are as provided in the general formula above, or as in the first through ninth embodiments.

In a eleventh embodiment of the invention, R⁴ is selected from methoxyethyl, pyridyl, pyridylmethyl, (1,2-dihydropyridyl)methyl, pyrimidylmethyl, oxetanylmethyl, (oxadiazolyl)methyl, (1,3,4-oxadiazolyl)methyl, triazolylmethyl, (1,2,4-triazolyl)methyl, (piperidinyl)methyl, pyridazinylmethyl, pyrazolylmethyl, tetrahydropyranylmethyl, (tetrahydro-2H-pyranyl)methyl, azetidinylmethyl, (1,2-dihydropyridinyl)methyl, pyrazinylmethyl, 3-methoxypropyl, morpholinylethyl, tetrahydro-2H-pyranyl, tetrahydropyranyl, pyridinyl, (tetrahydrofuranyl)methyl, oxetanylethyl, 3-(2-methoxyethoxy)propyl, 3-((2-2-methoxyethoxy)ethoxy)propyl, methoxypropyl, (methoxyethoxy)methylcarbonyl, 4-methoxybutylcarbonyl, and 3-(methoxypentoxy)propyl, wherein R⁴ is substituted with 0, 1, 2, or 3 R⁵ substituents and the other groups are as provided in the general formula above, or as in the first through ninth embodiments.

In a twelfth embodiment of the invention, R⁴ is selected from 3-(2-methoxyethoxy)propyl, 3-((2-2-methoxyethoxy)ethoxy)propyl, 3-(methoxypentoxy)propyl, piperidinylmethyl, methoxypropyl, and pyridylmethyl, wherein R⁴ is substituted with 0, 1, 2, or 3 R⁵ substituents and the other groups are as provided in the general formula above, or as in the first through ninth embodiments.

In a thirteenth embodiment of the invention, each R⁵ independently is selected from (C₁-C₆)alkylcarbonyl, hydroxy, —C(═O) (C₁-C₆)alkyl-(O—(C₁-C₆)alkyl)ₘ, —(C₁-C₆)alkyl-(O—(C₁-C₆)alkyl)ₘ, amino (C₁-C₆)akylcarbonyl, (C₁-C₆)alkylcarbonyl, and (C₁-C₆)alkylcarboxy, wherein each R⁵ independently is substituted with 0, 1, 2, or 2 R⁶ substituents and the other groups are as provided in the general formula above, or as in the first through twelfth embodiments.

In a fourteenth embodiment of the invention, each R⁵ independently is selected from (2-methoxyethoxy)methylcarbonyl, tert-butylcarboxy, 4-methoxybutylcarbonyl, methylcarbonyl, hydroxy,4-aminobutylcarbonyl, wherein each R⁵ independently is substituted with 0, 1, 2, or 3 R⁶ substituents and the other groups are as provided in the general formula above, or as in the first through twelfth embodiments.

In a fifteenth embodiment of the invention, each R⁶ is independently selected from amino, tert-butylcarboxy, methylcarboxy, ethylcarboxy, methyl, ethyl, propyl, isopropyl, fluoro, chloro and hydroxy and the other groups are as provided in the general formula above, or as in the first through fourteenth embodiments.

In a sixteenth embodiment of the invention, each R⁶ is independently amino, or tert-butylcarboxy and the other groups are as provided in the general formula above, or as in the first through fourteenth embodiments.

In a seventeenth embodiment of the invention, each n is independently 1, 2, or 3 and the other groups are as provided in the general formula above, or as in the first through fifteenth embodiments.

In an eighteenth embodiment of the invention, each m independently is 1, or 2 and the other groups are as provided in the general formula above, or as in the first through seventeenth embodiments.

In a nineteenth embodiment of the invention, R² is chloro, and R³ is chloro, and the other groups are as provided in the general formula Ia below, or as in the first through third and the tenth through eighteenth embodiments:

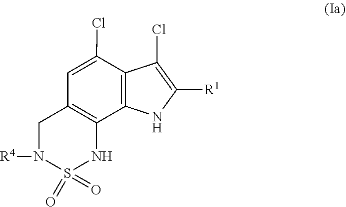

or a pharmaceutically acceptable salt thereof, wherein:
R¹ is selected from H, (C₁-C₆)alkyl, aminocarbonyl(C₁-C₆)alkyl, (C₁-C₆)alkylaminocarbonyl(C₀-C₆)alkyl, carboxy (C₁-C₆)alkyl, hydroxycarbonyl(C₀-C₆)alkyl), and (C₁-C₆)alkoxycarbonyl(C₀-C₆)alkyl);
R⁴ is selected from (C₁-C₆)alkoxy ((C₁-C₆)alkyl), heterocycloalkyl((C₀-C₆)alkyl), heteroaryl((C₀-C₆)alkyl), —C(═O) (C₁-C₆)alkyl-(O—(C₁-C₆)alkyl)ₙ, and —(C₁-C₆)alkyl-(O—(C₁-C₆))ₙ, wherein R⁴ is substituted with 0, 1, 2, or 3 R⁵ substituents;
each n is independently 1, 2, 3, or 4;
each R⁵ independently is selected from cyano, (C₁-C₆)haloalkyl, halogen, hydroxy, oxo, (C₁-C₆)alkyl, (C₁-C₆)alkoxy ((C₀-C₆)alkyl), —SO₂ (C₁-C₆)alkyl, amino, —C(═O) (C₁-C₆)alkyl-(O—(C₁-C₆)alkyl)ₘ, —(C₁-C₆)alkyl-(O—(C₁-C₆)alkyl)ₘ, amino (C₁-C₆)akylcarbonyl, (C₁-C₆)alkylcarboxy, (C₁-C₆)alkylcarbonyl, and —(C₁-C₆)alkylOH, wherein each R⁵ independently is substituted with 0, 1, 2, or 3 R⁶ substituents;
each R⁶ is independently selected from amino, (C₁-C₆)alkylcarboxy, (C₁-C₄)alkyl, halogen and hydroxy;
each m independently is 1, 2, or 3; and provided that when R⁴ is (C₁-C₆)alkoxy ((C₁-C₆)alkyl), heterocycloalkyl((C₀-C₆)alkyl), or heteroaryl((C₀-C₆)alkyl) then R¹ is other than hydrogen.

Non-limiting examples of the Compounds of Formula I include compounds 1-27 or a pharmaceutically acceptable salt thereof, as set forth in the Examples:
6,7-dichloro-3-(3-(2-methoxyethoxy)propyl)-1,3,4,9-tetrahydro-[1,2,6]thiadiazino[4,3-g]indole 2,2-dioxide;
6,7-dichloro-3-(3-(2-(2-methoxyethoxy)ethoxy)propyl)-1,3,4,9-tetrahydro-[1,2,6]thiadiazino[4,3-g]indole 2,2-dioxide;
6,7-dichloro-3-(3-((5-methoxypentyl)oxy)propyl)-1,3,4,9-tetrahydro-[1,2,6]thiadiazino[4,3-g]indole 2,2-dioxide;
1-(4-((6,7-dichloro-2,2-dioxido-4,9-dihydro-[1,2,6]thiadiazino[4,3-g]indol-3 (1H)-yl)methyl) piperidin-1-yl)-2-(2-methoxyethoxy)ethan-1-one;
1-(3-((6,7-dichloro-2,2-dioxido-4,9-dihydro-[1,2,6]thiadiazino[4,3-g]indol-3 (1H)-yl)methyl) piperidin-1-yl)-2-(2-methoxyethoxy)ethan-1-one;
1-(3-((6,7-dichloro-2,2-dioxido-4,9-dihydro-[1,2,6]thiadiazino[4,3-g]indol-3 (1H)-yl)methyl) piperidin-1-yl)-5-methoxypentan-1-one;
tert-butyl(5-(4-((6,7-dichloro-2,2-dioxido-4,9-dihydro-[1,2,6]thiadiazino[4,3-g]indol-3 (1H)-yl)methyl) piperidin-1-yl)-5-oxopentyl) carbamate;
5-amino-1-(4-((6,7-dichloro-2,2-dioxido-4,9-dihydro-[1,2,6]thiadiazino[4,3-g]indol-3 (1H)-yl)methyl) piperidin-1-yl) pentan-1-one;
6,7-dichloro-N-methyl-3-(piperidin-3-ylmethyl)-1,3,4,9-tetrahydro-[1,2,6]thiadiazino[4,3-g]indole-8-carboxamide 2,2-dioxide;
tert-butyl 3-((6,7-dichloro-8-(methylcarbamoyl)-2,2-dioxido-4,9-dihydro-[1,2,6]thiadiazino[4,3-g]indol-3 (1H)-yl)methyl) piperidine-1-carboxylate;
3-((1-acetylpiperidin-3-yl)methyl)-6,7-dichloro-N-methyl-1,3,4,9-tetrahydro-[1,2,6]thiadiazino[4,3-g]indole-8-carboxamide 2,2-dioxide;
methyl 6,7-dichloro-3-(3-methoxypropyl)-1,3,4,9-tetrahydro-[1,2,6]thiadiazino[4,3-g]indole-8-carboxylate 2,2-dioxide;
ethyl 6,7-dichloro-3-(3-methoxypropyl)-1,3,4,9-tetrahydro-[1,2,6]thiadiazino[4,3-g]indole-8-carboxylate 2,2-dioxide;
6,7-dichloro-3-(3-methoxypropyl)-N-methyl-1,3,4,9-tetrahydro-[1,2,6]thiadiazino[4,3-g]indole-8-carboxamide 2,2-dioxide;
6,7-dichloro-3-((2-hydroxypyridin-4-yl)methyl)-N-methyl-1,3,4,9-tetrahydro-[1,2,6]thiadiazino[4,3-g]indole-8-carboxamide 2,2-dioxide;
4-(6,7-dichloro-2,2-dioxido-3-(piperidin-3-ylmethyl)-1,3,4,9-tetrahydro-[1,2,6]thiadiazino[4,3-g]indol-8-yl)butanamide;
4-(6,7-dichloro-3-(3-methoxypropyl)-2,2-dioxido-1,3,4,9-tetrahydro-[1,2,6]thiadiazino[4,3-g]indol-8-yl)butanoic acid;
4-(6,7-dichloro-3-(3-methoxypropyl)-2,2-dioxido-1,3,4,9-tetrahydro-[1,2,6]thiadiazino[4,3-g]indol-8-yl)butanamide;
4-(3-((1-acetylpiperidin-3-yl)methyl)-6,7-dichloro-2,2-dioxido-1,3,4,9-tetrahydro-[1,2,6]thiadiazino[4,3-g]indol-8-yl)butanamide;
4-(6,7-dichloro-3-((2-hydroxypyridin-4-yl)methyl)-2,2-dioxido-1,3,4,9-tetrahydro-[1,2,6]thiadiazino[4,3-g]indol-8-yl)butanamide;
tert-butyl 3-((8-(4-amino-4-oxobutyl)-6,7-dichloro-2,2-dioxido-4,9-dihydro-[1,2,6]thiadiazino[4,3-g]indol-3 (1H)-yl)methyl) piperidine-1-carboxylate;
4-(6,7-dichloro-2,2-dioxido-3-(piperidin-3-ylmethyl)-1,3,4,9-tetrahydro-[1,2,6]thiadiazino[4,3-g]indol-8-yl)butanoic acid;
methyl 4-(6,7-dichloro-3-((2-hydroxypyridin-4-yl)methyl)-2,2-dioxido-1,3,4,9-tetrahydro-[1,2,6]thiadiazino[4,3-g]indol-8-yl)butanoate;
ethyl 4-(6,7-dichloro-3-((2-hydroxypyridin-4-yl)methyl)-2,2-dioxido-1,3,4,9-tetrahydro-[1,2,6]thiadiazino[4,3-g]indol-8-yl)butanoate; and
4-(6,7-dichloro-3-((2-hydroxypyridin-4-yl)methyl)-2,2-dioxido-1,3,4,9-tetrahydro-[1,2,6]thiadiazino[4,3-g]indol-8-yl)butanoic acid.

Unless defined otherwise, all technical and scientific terms used herein have the same meaning as is commonly understood by one of skill in the art to which this invention belongs.

As used in the specification and claims, the singular form “a”, “an” and “the” include plural references unless the context clearly dictates otherwise.

Unless defined otherwise, the term “Cx-Cy” when used in conjunction with a chemical moiety, such as alkyl, alkenyl, or alkynyl is meant to include groups that contain from x to y carbons in the chain. For example, the term “(Cx-Cy)alkyl” refers to substituted or unsubstituted saturated hydrocarbon groups, including straight-chain alkyl and branched-chain alkyl groups that contain from x to y carbons in the chain.

The term “C₀” or “C0″ as employed in expressions such as “(C₀-C₆)alkyl” and C0-6alkyl″ means a direct covalent bond; or when the term appears at the terminus of a substituent, C₀₋₆ alkyl means hydrogen. Similarly, when an integer defining the presence of a certain number of atoms in a group is equal to zero, it means that the atoms adjacent thereto are connected directly by a bond. For example, in the structure

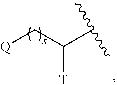

wherein s is an integer equal to zero, 1 or 2, the structure is

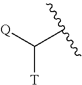

when s is zero.

The term “alkyl” as used herein refers to saturated linear or branched-chain monovalent hydrocarbon radicals. In one embodiment, an alkyl group contains from about 1 to about 10 carbon atoms. The term “(C₁-C₁₀)alkyl” as used herein refers to saturated linear or branched-chain monovalent hydrocarbon radicals of one to 10 carbon atoms, respectively. The term “(C₁-C₄)alkyl” as used herein refers to saturated linear or branched-chain monovalent hydrocarbon radicals of one to four carbon atoms, respectively. Illustrative examples of (C₁-C₄)alkyl include methyl, ethyl, 1-propyl, 2-propyl, I-butyl, 2-methyl-1-propyl, 2-butyl, and 2-methyl-2-propyl. The term (C₁-C₆)alkyl as used herein refers to saturated linear or branched-chain monovalent hydrocarbon radicals of one to six carbon atoms, respectively. Illustrative examples of (C₁-C₆)alkyl include, but are not limited to, methyl, ethyl, 1-propyl, 2-propyl, I-butyl, 2-methyl-1-propyl, 2-butyl, 2-methyl-2-propyl, 1-hexyl, 2-hexyl, 3-hexyl, 3,3-dimethylbutabyl, 2,2-dimethylbutanyl, 2,3-dimethylbutanyl, 2-methylpentanyl, 3-methylpentanyl, and 4-methylpentanyl.

The term “alkoxy” refers to an alkyl(carbon and hydrogen chain) group singularly bonded to oxygen (R—O). Non-limiting examples of alkoxy are methoxy (CH₃ O—)., ethoxy (CH₃ CH₂ O—) and butoxy (CH₃ CH₂ CH₂ O—).

The term “carbonyl” means a functional group composed of a carbon atom double-bonded to an oxygen atom, C═O.

The term “carboxy” or “carboxyl” means a carbon atom double bonded to an oxygen atom and single bonded to a hydroxyl group (—COOH).

“Cycloalkyl” or “C₃₋₁₂ cycloalkyl” means any univalent non-aromatic radicals derived from a monocyclic or bicyclic ring system having 3 to 12 ring carbons atoms and may be fully saturated, or practically unsaturated; said ring system may be (a) a C₃ to a C₈ monocyclic, fully saturated or partially unsaturated ring, or (b) a bicyclic ring. Here, the point of attachment for a “cycloalkyl” to the rest of the molecule is on the saturated ring. Bicyclic cycloalkyl ring systems include fused ring systems, where two rings share two atoms (e.g. decalin), spiro ring systems where two rings share one atom (e.g. spiro[4.5]decanyl) and bridge groups (e.g., norbornane).

Additional examples within the above meaning include, but are not limited to univalent radicals of cyclopropyl, cyclobutyl, cyclopentyl, cyclohexyl, cycloheptyl, bicyclo[2.2.2]octanyl, bicyclo[1.1.1]pentanyl, bicyclo[2.2.1]heptanyl, [1.1.1]-bicyclo pentane, 1-decalinyl, spiro[2.4]heptyl, spiro[2.2]pentyl, 2,3-dihydro-1H-indenyl, and norbornyl.

The term “C₃₋₈ cycloalkyl” (or “C₃-C₈ cycloalkyl”) means a cyclic ring of an alkane having three to eight total carbon atoms (i.e., cyclopropyl, cyclobutyl, cyclopentyl, cyclohexyl, cycloheptyl, or cyclooctyl). The terms “C₃₋₇ cycloalkyl”, “C₃₋₆ cycloalkyl”, “C₅₋₇ cycloalkyl” and the like have analogous meanings.

The term “(C₁-C₆) fluoroalkyl” as used herein refers to saturated linear or branched-chain monovalent hydrocarbon radicals of one to six carbon atoms substituted with one or more fluorine atoms. Illustrative examples include, but are not limited to, CHF₂, CH₂F, CF₃, CH₂CF₃, CF₂CH₃, CHFCH₃, CF(CH₃)₂, CH(CF₃)₂, CHFCF₃ and CF₂CF₃.

The term “(C₂-C₈)alkenyl” as used herein refers to straight or branched hydrocarbon chain radicals containing at least one double bond and having from two to five carbon atoms. A (C₂-C₈)alkenyl is attached to the rest of a molecule by a single bond through an sp² hybridized carbon. Illustrative examples of (C₂-C₈)alkenyl include, but are not limited to, ethenyl (i.e., vinyl), prop-1-enyl, but-1-enyl, pent-1-enyl, penta-1,4-dienyl.

The term “heteroaryl”, as used herein, represents a stable monocyclic, bicyclic or tricyclic ring system containing 5-14 carbon atoms and containing at least one ring heteroatom selected from N, S (including SO and SO₂) and O, wherein at least one of the heteroatom containing rings is aromatic. In the case of a heteroaryl ring system where one or more of the rings are saturated and contain one or more N atoms, the N can be in the form of quarternary amine. Bicyclic heteroaryl ring systems include fused ring systems, where two rings share two atoms, and spiro ring systems, where two rings share one atom. Heteroaryl groups within the scope of this definition include but are not limited to: azaindolyl, benzoimidazolyl, benzisoxazolyl, benzofuranyl, benzofurazanyl, benzopyrazolyl, benzotriazolyl, benzothiophenyl, benzothiazolyl, benzo[d]isothiazole, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, pyranyl, pyrazinyl, pyrazolyl, pyrrolyl, pyrazolopyrimidinyl, pyridazinyl, pyridyl, pyrimidyl, pyrimidinyl, pyrrolyl, quinazolinyl, quinolyl, quinoxalinyl, tetrazolyl, tetrazolopyridyl, thiadiazolyl, 5H-pyrrolo[3,4-b]pyridine, thiazolyl, thienyl, triazolyl, triazinyl, benzothiazolyl, benzothienyl, quinolinyl, quinazolinyl, and isoquinolinyl, and oxazolyl. If the heteroaryl contains nitrogen atoms, it is understood that the corresponding N-oxides thereof are also encompassed by this definition.

Examples of 5-6 member heteroaryls containing at least one ring heteroatom selected from N, S (including SO and SO₂) and O, include: furanyl, imidazolyl, isothiazolyl, isoxazolyl, oxadiazolyl, oxazolyl, oxazolinyl, isoxazolinyl, pyranyl, pyrazinyl, pyrazolyl, pyrrolyl, pyridazinyl, pyridyl, pyrimidyl, pyrimidinyl, pyrrolyl, tetrazolyl, thiadiazolyl, thiazolyl, thienyl, triazolyl, triazinyl, and oxazolyl.

The term “heterocycloalkyl,” as used herein, refers to a non-aromatic saturated monocyclic or multicyclic ring system comprising 3 to about 14 ring atoms, wherein from 1 to 4 of the ring atoms are independently O, S, or N, and the remainder of the ring atoms are carbon atoms. There are no adjacent oxygen and/or sulfur atoms present in the ring system. A heterocycloalkyl group can be joined via a ring carbon or ring nitrogen atom. Said ring system may be (a) a saturated monocyclic ring or a partially unsaturated ring, or (b) a bicyclic ring system having at one saturated ring with at least one ring atom that is independently O, S, or N. The other ring of the bicyclic system (b) may be saturated or partially unsaturated. For a bicyclic system, the rings are fused across two adjacent ring carbon atoms (e.g., decahydroisoquinoline, 2,3-dihydro-1H-benzo[d]imidazolyl, isoindolinyl), at one ring carbon atom (e.g., 1,4-dioxaspiro[4.5]decane), or are bridged groups (e.g., 2,5-diazabicyclo[2.2.1]heptyl, quinuclidinyl).

In one embodiment, a heterocycloalkyl group is monocyclic and has from about 3 to about 7 ring atoms. In another embodiment, a heterocycloalkyl group is monocyclic has from about 5 to about 8 ring atoms. In another embodiment, a heterocycloalkyl group is bicyclic and has from about 8 to about 11 ring atoms. In still another embodiment, a heterocycloalkyl group is monocyclic and has 5 or 6 ring atoms. In one embodiment, a heterocycloalkyl group is monocyclic. In another embodiment, a heterocycloalkyl group is bicyclic. There are no adjacent oxygen and/or sulfur atoms present in the ring system. Non-limiting examples of monocyclic heterocycloalkyl rings include oxetanyl, piperidyl, pyrrolidinyl, piperazinyl, morpholinyl, thiomorpholinyl, thiazolidinyl, 1,4-dioxanyl, tetrahydrofuranyl, tetrahydropyranyl, tetrahydrothiophenyl, 1,2-dihydropyridyl(1,2-dihydropyridinyl), beta lactam, gamma lactam, delta lactam, beta lactone, gamma lactone, delta lactone, and pyrrolidinone, and oxides thereof and all isomers thereof.

The term “phenyl” as used herein refers to a radical with the formula C₆H₅. The term “halogen” includes fluoro, chloro, bromo and iodo.

The term “oxy” means an oxygen (O) atom. The term “thio” means a sulfur(S) atom.

The term “oxo” means “=O”. The term “carbonyl” means “C═O.”

The term “substituted” as used herein refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

The term “optional” or “optionally” as used herein means that the subsequently described event of circumstances may or may not occur, and that the description includes instances where the event or circumstance occurs and instances in which it does not. For example, “optionally substituted aryl” means that the aryl group may or may not be substituted and that the description includes both substituted aryl groups and aryl groups having no substitution.

By “pharmaceutically acceptable” is meant that the ingredients of the pharmaceutical composition must be compatible with each other and not deleterious to the recipient thereof.

Where any amine is present in the compound, the N atom may be optionally in the form of a quaternary amine having one or more appropriate additional substitutions, as further described herein.

When any variable (e.g., n, Ra, Rb, etc.) occurs more than one time in any constituent or in Formula (I), its definition on each occurrence is independent of its definition at every other occurrence. Also, combinations of substituents and/or variables are permissible only if such combinations result in stable compounds.

When any ring atom is specified as being optionally substituted with, or in a specified form, for example, S substituted with oxo groups, or N in the form of a N-oxide, this does not preclude the substitution of any ring atom with the other listed optional substituents when not substituted with oxo groups or in the form of a N-oxide.

“Celite R” (Fluka)diatomite is diatomaceous earth, and can be referred to as “celite”.

By “stable compound” or “stable structure” is meant a compound that is sufficiently robust to survive isolation to a useful degree of purity from a reaction mixture, and formulation into an efficacious therapeutic agent. The compounds of the present invention are limited to stable compounds embraced by Formula (I).

The term “compound” refers to the compound and, in certain embodiments, to the extent they are stable, any hydrate or solvate thereof. A hydrate is the compound complexed with water, and a solvate is the compound complexed with an organic solvent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

Lines drawn into the ring systems from substituents indicate that the indicated bond can be attached to any of the substitutable ring atoms. If the ring system is polycyclic, it is intended that the bond be attached to any of the suitable carbon atoms on the proximal ring only.

Under standard nomenclature used throughout this disclosure, the terminal portion of the designated side chain is preceded by the adjacent functionality toward the point of attachment. For example, a C₁-5 alkylcarbonylamino C₁-6 alkyl substituent is equivalent to

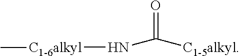

Structural representations of compounds having substituents terminating with a methyl group may display the terminal methyl group either using the characters “CH3”, e.g. “—CH3” or using a straight line representing the presence of the methyl group, e.g. “ [symbol] ”, i.e., “

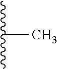

” and “ [symbol] ” have equivalent meanings.

For variable definitions containing terms having repeated terms, e.g., (CRiRj)r, where r is the integer 2, Ri is a defined variable, and Rj is a defined variable, the value of Ri may differ in each instance in which it occurs, and the value of Rj may differ in each instance in which it occurs. For example, if Ri and Rj are independently selected from the group consisting of methyl, ethyl, propyl and butyl, then (CRiRj)2 can be

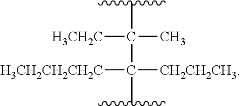

Unless expressly stated to the contrary, all ranges cited herein are inclusive. For example, a heteroaromatic ring described as containing from “1 to 4 heteroatoms” means the ring can contain, 1, 2, 3 or r heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, 3 heteroatoms, and 4 heteroatoms. Similarly, C₁-C₆ when used with a chain, for example an alkyl chains means that the chain can contain 1, 2, 3, 4, 5, or 6 carbon atoms. It also includes all ranges contained therein including C₁-C₅, C₁-C₄, C₁-C₃, C₁-C₂, C₂-C₆, C₃-C₆, C₄-C₆, C₅-C₆, and all other possible combinations.

In choosing compounds of the present invention, one of ordinary skill in the art will recognize that the various substituents, i.e. R¹, RA, etc., are to be chosen in conformity with well-known principles of chemical structure connectivity and stability.

As used herein, the term “composition” is intended to encompass a product comprising the specified ingredients in the specified amounts, as well as any product which results from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987)₁₄ of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide a compound of Formula (I) or a pharmaceutically acceptable salt of the compound. The transformation may occur by various mechanisms (e.g., by metabolic or chemical processes), such as, for example, through hydrolysis in blood. For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino (C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino (C₂-C₃)alkyl, and the like.

Similarly, if a compound of Formula (I) contains an alcohol functional group, a prodrug can be formed by the replacement of one or more of the hydrogen atoms of the alcohol groups with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino (C₁-C₄)alkyl, α-amino (C₁-C₄)alkylene-aryl, arylacyl and α-aminoacyl, or α-aminoacyl-α-aminoacyl, where each α-aminoacyl group is independently selected from the naturally occurring L-amino acids, or glycosyl (the radical resulting from the removal of a hydroxyl group of the hemiacetal form of a carbohydrate).

If a compound of Formula (I) incorporates an amine functional group, a prodrug can be formed by the replacement of a hydrogen atom in the amine group with a group such as, for example, R-carbonyl-, RO-carbonyl-, NRR′-carbonyl- wherein R and R′ are each independently (C₁-C₁₀)alkyl, (C₃-C₇) cycloalkyl, benzyl, a natural a aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl; carboxy (C₁-C₆)alkyl; amino (C₁-C₄)alkyl or mono-N— or di-N,N—(C₁-C₆)alkylaminoalkyl; —C(Y⁴)Y⁵ wherein Y⁴ is H or methyl and Y⁵ is mono-N— or di-N,N—(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (e.g., methyl, ethyl, n-propyl, isopropyl, t-butyl, sec-butyl or n-butyl), alkoxyalkyl (e.g., methoxymethyl), aralkyl (e.g., benzyl), aryloxyalkyl (for example, phenoxymethyl), aryl (e.g., phenyl optionally substituted with, for example, halogen, C₁₄alkyl, —O—(C₁₄alkyl) or amino); (2) sulfonate esters, such as alkyl- or aralkylsulfonyl (for example, methanesulfonyl); (3) amino acid esters, including those corresponding to both natural and non-natural amino acids (e.g., L-valyl or L-isoleucyl); (4)phosphonate esters and (5) mono-, di- or triphosphate esters. The phosphate esters may be further esterified by, for example, a C₁₋₂₀ alcohol or reactive derivative thereof, or by a 2,3-di(C₆₋₂₄) acyl glycerol.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more compounds of the invention may optionally be converted to a solvate. Preparation of solvates is generally known. Thus, for example, M. Caira et al, J. Pharmaceutical Sci., 93 (3), 601-611 (2004) describe the preparation of the solvates of the antifungal fluconazole in ethyl acetate as well as from water. Similar preparations of solvates, hemisolvates, hydrates and the like are described by E. C. van Tonder et al, AAPS PharmSciTech., 5 (1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

The compound of Formula (I) can form salts which are also within the scope of this invention. Reference to a compound of Formula (I) herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Formula (I) may be formed, for example, by reacting a compound of Formula (I) with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66 (1)1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), arylalkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures can be separated into their individual diastereomers on the basis of their physical chemical differences by methods well-known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the compound of Formula (I) may be atropisomers (e.g., substituted biaryls) and are considered as part of this invention. Enantiomers can also be directly separated using chiral chromatographic techniques.

It is also possible that the compound of Formula (I) may exist in different tautomeric forms, and all such forms are embraced within the scope of the invention. For example, all keto-enol and imine-enamine forms of the compounds are included in the invention.

Unless otherwise indicated, all stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. If a compound of Formula (I) incorporates a double bond or a fused ring, both the cis- and trans-forms, as well as mixtures, are embraced within the scope of the invention.

When a substituent on a chiral carbon atom is depicted without specific stereochemistry (by using a straight line bond to a chiral center), it is to be understood that both the alpha and beta configurations of said substituent group are to be considered part of the present invention.

In the Examples section below, compounds of the present invention that have been purified as individual stereoisomers are sometimes depicted in non-stereospecific form but identified using one or more of the terms: “diastereomer 1,” “diastereomer 2,” “isomer 1,” “isomer 2,” “first eluding enantiomer”, “enantiomer A” and “enantiomer B.” In this instance, the absolute stereochemistry of each isolated diastereomer and enantiomeric center has not been determined and the terms used above are used to represent each individual purified stereochemically pure compound.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, racemates or prodrugs of the inventive compounds.

In the Compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula (I). For example, different isotopic forms of hydrogen (H) include protium (1H) and deuterium (2H). Protium is the predominant hydrogen isotope found in nature. Enriching for deuterium may provide certain therapeutic advantages, such as increasing in vivo half-life or reducing dosage requirements, or may provide a compound useful as a standard for characterization of biological samples. Isotopically-enriched Compounds of Formula (I) can be prepared without undue experimentation by conventional techniques well known to those skilled in the art or by processes analogous to those described in the Schemes and Examples herein using appropriate isotopically-enriched reagents and/or intermediates. In one embodiment, a Compound of Formula (I) has one or more of its hydrogen atoms replaced with deuterium.

In another embodiment, the Compounds of Formula (I) are in substantially purified form.

The term “effective amount” or “therapeutically effective amount” refers to that amount of a compound described herein that is sufficient to affect the intended application, including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried. It is recognized that one skilled in the art may affect the cancerous disorders by treating a patient presently afflicted with the disorders or by prophylactically treating a patient afflicted with such disorders with an effective amount of the compound of the present invention.

As used herein, “treatment”, “treatment of” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “degrader” as used herein refers to a compound having the ability to induce the degradation of a target protein. For example, a degrader may induce the ubiquitination and subsequent proteasomal degradation of a target protein. Targeted protein degradation can be undertaken for the purposes of inhibiting the biological function of the target protein, meaning that a degrader can be a member of a specific sub-class of antagonist. Compounds that potentiate the formation of a complex between a target protein and any portion of an E3 ubiquitin ligase complex are specifically included within this definition.

The term “RBM39 degrader” as used herein refers to a compound, such as a compound of Formula I or II, having the ability to induce the degradation of RBM39 protein. For example, an RBM39 degrader may induce the ubiquitination and subsequent proteasomal degradation of RBM39 protein. Targeted protein degradation can be undertaken for the purposes of inhibiting the biological function of the target protein. Compounds that potentiate the formation of a complex between RBM39 protein and any portion of an E3 ubiquitin ligase complex are included within this definition.

The term “cell proliferation” refers to a phenomenon by which the cell number has changed as a result of division. This term also encompasses cell growth by which the cell morphology has changed (e.g., increased in size) consistent with a proliferative signal.

The term “subject” refers to an animal, such as a mammal, for example a human. The methods described herein can be useful in both human therapeutics and veterinary applications. In some embodiments, the subject is a mammal, and in some embodiments, the subject is human.

“Mammal” includes humans and both domestic animals such as laboratory animals and household pets (e.g., cats, dogs, swine, cattle, sheep, goats, horses, rabbits), and non-domestic animals such as wildlife and the like.

The term “in vivo” refers to an event that takes place in a subject's body.

The term “in vitro” refers to an event that takes places outside of a subject's body. For example, an in vitro assay encompasses any assay run outside of a subject. In vitro assays encompass cell-based assays in which cells alive or dead are employed. In vitro assays also encompass a cell-free assay in which no intact cells are employed.

The disclosure is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure includes compounds produced by a process comprising administering a compound of this disclosure to a mammal for a period of time sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood or other biological samples.

The term “substantially pure” means that the isolated material is at least 90% pure, and preferably 95% pure, and even more preferably 99% pure as assayed by analytical techniques known in the art.

For purposes of this specification, the following abbreviations have the indicated meanings:
Ac acetyl
ACN acetonitrile
AcO acetate
BOC t-butyloxycarbonyl
CBZ carbobenzoxy
CDI carbonyldiimidazole
DBU 1,8-Diazabicycloundec-7-ene
DCC 1,3-dicyclohexylcarbodiimide
DCE 1,2-dichloroethane
DCM dichloromethane
(dF(CF₃)ppy) 2-(2,4-difluorophenyl)-5-trifluoromethylpyridine
DIAD Diisopropyl azodicarboxylate
DIBAL diisobutyl aluminum hydride
DIPEA or DIEA N,N-diisoproylethylamine, also known as Hunig's base
DMA dimethylacetamide
DMAP 4-(dimethylamino)pyridine
DMF dimethylformamide
DMF·DMA N,N-dimethylformamide dimethyl acetal
DMP Dess-Martin periodinane
DPPA Diphenylphosphoryl azide
DPPP 1,3-bis(diphenylphosphino) propane
Dtbbpy 4,4′-di-tert-butyl-2,2′-dipyridyl
EDC or EDCl 1-(3-dimethylaminopropyl)-3-ethylcarbodiimide
Hydrochloride
EDCl 1-ethyl-3-(3-dimethylaminopropyl) carbodiimide
EDTA ethylenediaminetetraacetic acid, tetrasodium salt
EtOAc ethyl acetate
FMOC 9-fluorenylmethoxycarbonyl
HMPA hexamethylphosphoramide
HATU O-(7-Azabenzotriazol-1-yl)-N,N,N′,N′-tetramethyluronium hexafluorophosphate
HOAt 1-Hydroxy-7-azabenzotriazole or 3H-[1,2,3]triazolo[4,5-b]pyridin-3-ol
HOBt 1-hydroxybenzotriazole
HRMS high resolution mass spectrometry
IBCF isobutyl chloroformate
KHMDS potassium hexamethyldisilazane
LC-MS Liquid chromatography-mass spectrometry
LDA lithium diisopropylamide
LiHMDS lithium hexamethyldisilazane
MCPBA metachloroperbenzoic acid
MMPP magnesium monoperoxyphthlate hexahydrate
Ms methanesulfonyl=mesyl
MsO methanefulfonate=mesylate
MTBE methyl t-butyl ether
NBS N-bromosuccinimide
NCS N-chlorosuccinimide
NIS N-iodosuccinimide
NMM 4-methylmorpholine
NMP N-methylpyrrolidinone
NMR Nuclear magnetic resonance
PCC pyridinium chlorochromate
PDC pyridinium dichromate
Ph phenyl
PPTS pyridinium p-toluene sulfonate
pTSA p-toluene sulfonic acid
PyH·Br3 pyridine hydrobromide perbromide
r.t./RT room temperature
rac. Racemic
SEM [2-(trimethylsilyl)ethoxy]methyl
T3P 2,4,6-Tripropyl-1,3,5,2,4,6-trioxatriphosphinane 2,4,6-trioxide
TBAF tetrabutylammonium fluoride
TEA triethylamine
TFA trifluoroacetic acid
TFAA trifluoroacetic acid anhydride
TfO trifluoromethanesulfonate=triflate
THF tetrahydrofuran
TLC thin layer chromatography
TMSCl trimethylsilyl chloride

The present invention is directed to a compound of the present invention or a pharmaceutically acceptable salt thereof for use in medicine. The present invention is further directed to a use of a compound of the present invention or a pharmaceutically acceptable salt thereof for the manufacture of a medicament for treating a disorder associated with RBM39 degradation function in a mammalian patient in need thereof. The present invention is further directed to a use of a compound of the present invention or a pharmaceutically acceptable salt thereof for the manufacture of a medicament for treating a disorder associated with cancers, such as, for example, acute myeloid leukemia (AML), colon, EZH2 mutant limphomas, and melanomia in a mammalian patient in need thereof.

The present invention is further directed to a use of the compound of formula 1 or a pharmaceutically acceptable salt thereof, in the preparation of a medicament for modulating at least one activity selected from RBM39 activity and DCAF15 activity, in a patient in need thereof. In one embodiment, the activity is RBM39 activity. In another embodiment of the invention, the activity is DCAF15 activity. In yet another embodiment of the invention, the modulation included both RBM39 activity and DCAF 15 activity where RBM39 protein degradation is the result of compound modulating (binding to) DCAF15 and recruiting RBM39.

The subject compounds may be further useful in a method for the prevention, treatment, control, amelioration, or reduction of risk of the diseases, disorders and conditions noted herein. The subject compounds are further useful in a method for the prevention, treatment, control, amelioration, or reduction of risk of the aforementioned diseases, disorders and conditions in combination with other agents.

The compounds of the present invention may be used in combination with one or more other drugs in the treatment, prevention, control, amelioration, or reduction of risk of diseases or conditions for which compounds of the present invention or the other drugs may have utility, where the combination of the drugs together are safer or more effective than either drug alone. Such other drug(s) may be administered, by a route and in an amount commonly used therefore, contemporaneously or sequentially with a compound of the present invention. When a compound of the present invention is used contemporaneously with one or more other drugs, a pharmaceutical composition in unit dosage form containing such other drugs and the compound of the present invention may be desirable. However, the combination therapy may also include therapies in which the compound of the present invention and one or more other drugs are administered on different overlapping schedules. It is also contemplated that when used in combination with one or more other active ingredients, the compounds of the present invention and the other active ingredients may be used in lower doses than when each is used singly.

Accordingly, the pharmaceutical compositions of the present invention include those that contain one or more other active ingredients, in addition to a compound of the present invention. The above combinations include combinations of a compound of the present invention not only with one other active compound, but also with two or more other active compounds. Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefore, contemporaneously or sequentially with a compound of the present invention. Accordingly, the pharmaceutical compositions of the present invention include those that also contain one or more other active ingredients, in addition to a compound of the present invention. The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, such as about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

Accordingly, the subject compounds may be used alone or in combination with other agents which are known to be beneficial in the subject indications or other drugs that affect receptors or enzymes that either increase the efficacy, safety, convenience, or reduce unwanted side effects or toxicity of the compounds of the present invention. The subject compound and the other agent may be co-administered, either in concomitant therapy or in a fixed combination.

In one embodiment, the subject compound may be employed in combination with anti-Alzheimer's agents, AChEis (Aricept (donepezil)) and NMDA blocker Namenda (memantine), beta-secretase inhibitors, gamma-secretase inhibitors, HMG-COA reductase inhibitors, NSAID's including ibuprofen, vitamin E, and anti-amyloid antibodies.

One or more additional pharmacologically active agents may be administered in combination with a compound of Formula (I) (or a pharmaceutically acceptable salt thereof). An additional active agent (or agents) is intended to mean a pharmaceutically active agent (or agents) that is active in the body, including pro-drugs that convert to pharmaceutically active form after administration, which are different from the compounds of Formula (I). The additional active agents also include free-acid, free-base and pharmaceutically acceptable salts of said additional active agents. Generally, any suitable additional active agent or agents, including chemotherapeutic agents or therapeutic antibodies may be used in any combination with a compound of Formula (I) in a single dosage formulation (a fixed dose drug combination), or in one or more separate dosage formulations which allows for concurrent or sequential administration of the active agents (co-administration of the separate active agents) to subjects. In addition, the compounds of Formulae (I) (or pharmaceutically acceptable salts thereof) can be administered in combination with radiation therapy, hormone therapy, surgery or immunotherapy.

The present application also provides methods for combination therapies in which the additional active agent is known to modulate other pathways, or other components of the same pathway, or even overlapping sets of target enzymes which are used in combination with a compound of Formula (I), or a pharmaceutically acceptable salt thereof. In one embodiment, such therapy includes but is not limited to the combination of one or more compounds of Formula (I) with chemotherapeutic agents, therapeutic antibodies, and radiation treatment, to provide a synergistic or additive therapeutic effect.

In one embodiment, the combination therapies comprise chemotherapeutic agents. Many such agents are presently known in the art and can be used in combination with the compounds of Formula (I). In some embodiments, the chemotherapeutic agent is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. Non-limiting examples are cytotoxic agents, and non-peptide small molecules such as Gleevec® (Imatinib Mesylate), Kyprolis® (carfilzomib), Velcade® (bortezomib), Casodex (bicalutamide), Iressa® (gefitinib), and Adriamycin as well. Non-limiting examples of chemotherapeutic agents include alkylating agents such as thiotepa and cyclosphosphamide (CYTOXANTM™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™ chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifiuridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel and docetaxel; retinoic acid; esperamicins; capecitabine; and pharmaceutically acceptable salts, acids or derivatives of any of the above.

Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4 (5)-imidazoles, 4-hydroxy tamoxifen, trioxifene, keoxifene, LY 117018, onapristone, and toremifene; and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); and topoisomerase inhibitor RFS 2000.

Where desired, the compounds of Formula (I) or pharmaceutical compositions containing such compounds can be used in combination with commonly prescribed anti-cancer drugs such as Herceptin®, Avastin®, Erbitux®, Rituxan®, Taxol®, Arimidex®, Taxotere®, ABVD, AVIClNE, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, Antineoplastic, antitumorigenic herbs, apaziquone, atiprimod, azathioprine, belotecan, bendamustine, BIBW 2992, biricodar, brostallicin, bryostatin, buthionine sulfoximine, calyculin, cell-cycle nonspecific antineoplastic agents, dichloroacetic acid, discodermolide, elsamitrucin, enocitabine, epothilone, eribulin, everolimus, exatecan, exisulind, ferruginol, forodesine, fosfestrol, ICE chemotherapy regimen, IT-101, imexon, imiquimod, indolocarbazole, irofulven, laniquidar, larotaxel, lenalidomide, lucanthone, lurtotecan, mafosfamide, mitozolomide, nafoxidine, nedaplatin, olaparib, ortataxel, PAC-1, pixantrone, proteasome inhibitor, rebeccamycin, resiquimod, rubitecan, SN-38, salinosporamide a, sapacitabine, swainsonine, talaporfin, tariquidar, tegafur-uracil, temozolimide, tesetaxel, triplatin tetranitrate, tris(2-chloroethyl)amine, troxacitabine, Vadimezan, Vinflunine, ZD6126 or Zosuquidar.

The present application further provides a method for using the compounds of Formula (I) or pharmaceutical compositions provided herein, in combination with radiation therapy for inhibiting abnormal cell growth or treating the hyperproliferative disorder in the mammal. Techniques for administering radiation therapy are known in the art, and these techniques can be used in the combination therapy described herein. The administration of the compound of Formula (I) in this combination therapy can be determined as described herein.

Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachy therapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g., At-211, I-131, 1-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as I-125, 1-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive microspheres.

The compounds of Formula (I) or pharmaceutical compositions containing such compounds can be used in combination with an amount of one or more substances selected from anti-angiogenesis agents, signal transduction inhibitors, antiproliferative agents, glycolysis inhibitors, or autophagy inhibitors.

Anti-angiogenesis agents, such as MMP-2 (matrix-metalloproteinase 2) inhibitors and MMP-9 (matrix-metalloproteinase 9) inhibitors, can be used in conjunction with a compound of the disclosure and pharmaceutical compositions described herein. Anti-angiogenesis agents include, for example, rapamycin, temsirolimus (CCI-779), everolimus (RAD001), sorafenib, sunitinib, and bevacizumab. Examples of useful matrix metalloproteinase inhibitors are described in WO 96/33172, WO 96/27583 European Patent Publication No. EP0818442, European Patent Publication No. EP1004578, WO 98/07697, WO 98/03516, WO 98/34918, WO 98/34915, WO 98/33768, WO 98/30566, European Patent Publication No. 606046, European Patent Publication No. 931788, WO 90/05719, WO 99/52910, WO 99/52889, WO 99/29667, WO 1999007675, European Patent Publication No. EP1786785, European Patent Publication No. EP1181017, U.S. Publication No. US20090012085, U.S. Pat. Nos. 5,863,949, 5,861,510, and European Patent Publication No. EP0780386. Preferred MMP-2 and MMP-9 inhibitors are those that have little or no activity inhibiting MMP-1. More preferred, are those that selectively inhibit MMP-2 and/or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors useful in the combinations are AG-3340, RO 32-3555, and RS 13-0830.

The compounds of Formula (I) may also be used in co-therapies with other anti-neoplastic agents, such as acemannan, aclarubicin, aldesleukin, alemtuzumab, alitretinoin, altretamine, amifostine, aminolevulinic acid, amrubicin, amsacrine, anagrelide, anastrozole, ANCER, ancestim, ARGLABIN, arsenic trioxide, BAM 002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-N1, interferon alfa-n3, interferon alfacon-1, interferon alpha, natural, interferon beta, interferon beta-1a, interferon beta-1b, interferon gamma, natural interferon gamma-1a, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 63 1570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, VIRULIZIN, zinostatin stimalamer, or zoledronic acid; abarelix; AE 941 (Aeterna), ambamustine, bcl-2 (Genta), APC 8015 (Dendreon), cetuximab, decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

The compounds of Formula (I) may further be used with VEGFR inhibitors.

In some embodiments, the combination comprises a composition of the present invention in combination with at least one anti-angiogenic agent. An agent can be an agonist, antagonist, allosteric modulator, toxin or, more generally, may act to inhibit or stimulate its target (e.g., receptor or enzyme activation or inhibition), and thereby promote cell death or arrest cell growth.

Exemplary anti-angiogenic agents include ERBITUX™, KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF, or soluble VEGF receptors or a ligand binding region thereof) such as AVASTIN™ or VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as Vectibix (panitumumab), IRES S A™ (gefitinib), TARCEVA™ (erlotinib), anti-Ang1 and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). The pharmaceutical compositions of the present invention can also include one or more agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor “c-met”. Other anti-angiogenic agents include Campath, IL-8, B-FGF, Tek antagonists (Ceretti et al, U.S. Publication No. 2003/0162712; U.S. Pat. No. 6,413,932), anti-TWEAK agents (e.g., specifically binding antibodies or antigen binding regions, or soluble TWEAK receptor antagonists; see, Wiley, U.S. Pat. No. 6,727,225), ADAM distintegrin domain to antagonize the binding of integrin to its ligands (Fanslow et al., U.S. Publication No. 2002/0042368), specifically binding anti-eph receptor and/or anti-ephrin antibodies or antigen binding regions (U.S. Pat. Nos. 5,981,245; 5,728,813; 5,969, 110; 6,596,852; 6,232,447; and 6,057,124), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto).

Additional anti-angiogenic/anti-tumor agents include: SD-7784 (Pfizer, USA); cilengitide (Merck KGaA, Germany); pegaptanib octasodium, (Gilead Sciences, USA); alphastatin (BioActa, UK); M-PGA, ilomastat, (Arriva, USA); emaxanib, (Pfizer, USA); vatalanib (Novartis, Switzerland); 2-methoxyestradiol; TLC ELL-12 (Elan, Ireland); anecortave acetate (Alcon, USA); alpha-D148 Mab, (Amgen, USA); CEP-7055 (Cephalon, USA); anti-Vn Mab (Crucell, Netherlands) angiocidin (InKine Pharmaceutical, USA); KM-2550 (Kyowa Hakko, Japan); SU-0879 (Pfizer, USA); CGP-79787 (Novartis, Switzerland, EP 970070); fibrinogen-E fragment (BioActa, UK); TBC-1635 (Encysive Pharmaceuticals, USA); SC-236 (Pfizer, USA); metastatin (EntreMed, USA); maspin (Sosei, Japan); ER-68203-00 (IVAX, USA); benefin (Lane Labs, USA); Tz-93 (Tsumura, Japan); TAN-1120 (Takeda, Japan); FR-111142 (Fujisawa, Japan); platelet factor 4; vascular endothelial growth factor antagonist, (Borean, Denmark); bevacizumab (pINN), (Genentech, USA); angiogenesis inhibitors, (SUGEN, USA); XL 784, (Exelixis, USA); XL 647, (Exelixis, USA); MAb, alpha5beta3 integrin, second generation, (Applied Molecular Evolution, USA and MedImmune, USA); enzastaurin hydrochloride (USAN), (Lilly, USA); CEP 7055, (Cephalon, USA and Sanofi-Synthelabo, France); BC 1, (Genoa Institute of Cancer Research, Italy); rBPI 21 and BPI-derived antiangiogenic (XOMA, USA); PI 88 (Progen, Australia); cetuximab, (Aventis, France); AVE 8062 (Ajinomoto, Japan); AS 1404, (Cancer Research Laboratory, New Zealand); SG 292, (Telios, USA); endostatin, (Boston Childrens Hospital, USA); ANGIOSTATIN (Boston Childrens Hospital, USA); AZD 6474, (AstraZeneca, UK); ZD 6126 (Angiogene Pharmaceuticals, UK); PPI 2458, (Praecis, USA); AZD 9935 (AstraZeneca, UK); AZD 2171 (AstraZeneca, UK); vatalanib (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); SDX 103, (University of California at San Diego, USA); PX 478, (ProlX, USA); METASTATIN (EntreMed, USA); troponin I, (Harvard University, USA); SU 6668, (SUGEN, USA); OXI 4503 (OXIGENE, USA); motuporamine C, (British Columbia University, Canada); CDP 791 (Celltech Group, UK); atiprimod (GlaxoSmithKline, UK); E 7820 (Eisai, Japan); CYC 381 (Harvard University, USA); AE 941 (Aeterna, Canada); urokinase plasminogen activator inhibitors; HIF-1 alfa inhibitors; angiocidin (InKine, USA); GW 2286 (GlaxoSmithKline, UK); EHT 0101 (ExonHit, France); CP 868596 (Pfizer, USA); CP 564959 (OSI, USA); CP 547632 (Pfizer, USA); 786034, (GlaxoSmithKline, UK); KRN 633 (Kirin Brewery, Japan); tumor necrosis factor-alpha inhibitors; KDR kinase inhibitors; combretastatin A4 prodrug (Arizona State University, USA); chondroitinase AC (IBEX, Canada); BAY RES 2690 (Bayer, Germany); tetrathiomolybdate (University of Michigan, USA); GCS 100 (Wayne State University, USA) CV 247 (Ivy Medical, UK); CKD 732, (Chong Kun Dang, South Korea); MAb, vascular endothelium growth factor, (Xenova, UK); irsogladine (Nippon Shinyaku, Japan); RG 13577 (Aventis, France); VE-cadherin-2 antagonists; vasostatin, (National Institutes of Health, USA); Flk-1, (ImClone Systems, USA); TZ 93 (Tsumura, Japan); TumStatin (Beth Israel Hospital, USA); forms of FLT 1 (vascular endothelial growth factor receptor 1); Tie-2 ligands (Regeneron, USA); and thrombospondin 1 inhibitor (Allegheny Health, USA).

Additional active compounds/agents that can be used in the treatment of cancers and that can be used in combination with one or more compounds of Formula (I) include: epoetin alfa; darbepoetin alfa; panitumumab; pegfilgrastim; palifermin; filgrastim; denosumab; ancestim or a pharmaceutically acceptable salt thereof.

The compounds of the present invention may also be used in combination with an additional pharmaceutically active compound that disrupts or inhibits RAS-RAF-ERK or PI3K-AKT-TOR signaling pathways. In other such combinations, the additional pharmaceutically active compound is a PD-1 and PD-L1 antagonist. The compounds or pharmaceutical compositions of the disclosure can also be used in combination with an amount of one or more substances selected from EGFR inhibitors, MEK inhibitors, ERK inhibitors, PI3K inhibitors, AKT inhibitors, TOR inhibitors, Mcl-1 inhibitors, BCL-2 inhibitors, SHP2 inhibitors, proteasome inhibitors, and immune therapies, including monoclonal antibodies, immunomodulatory imides (IMiDs), anti-PD-1, anti-PDL-1, anti-CTLA4, anti-LAG1, and anti-OX40 agents, GITR agonists, CAR-T cells, and BiTEs.

EGFR inhibitors include, but are not limited to, small molecule antagonists, antibody inhibitors, or specific antisense nucleotide or siRNA. Useful antibody inhibitors of EGFR include cetuximab (Erbitux), panitumumab (Vectibix), zalutumumab, nimotuzumab, and matuzumab. Small molecule antagonists of EGFR include gefitinib, erlotinib, and lapatinib.

Antibody-based EGFR inhibitors include any anti-EGFR antibody or antibody fragment that can partially or completely block EGFR activation by its natural ligand. Non-limiting examples of antibody-based EGFR inhibitors include those described in Modjtahedi, H., et al., 1993, Br. J. Cancer 67:247-253; Teramoto, T., et al., 1996, Cancer 77:639-645; Goldstein et al, 1995, Clin. Cancer Res. 1:1311-1318; Huang, S. M., et al., 1999, Cancer Res. 15: 59 (8): 1935-40; and Yang, X., et al., 1999, Cancer Res. 59:1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

MEK inhibitors include, but are not limited to, CI-1040, AZD6244, PD318088, PD98059, PD334581, RDEA119, ARRY-142886, ARRY-438 162, and PD-325901.

PI3K inhibitors include, but are not limited to, wortmannin, 17-hydroxywortmannin analogs described in WO 06/044453, 4-[2-(1H-Indazol-4-yl)-6-[[4-(methylsulfonyl) piperazin-1-yl]methyl]thieno[3,2-d]pyrimidin-4-yl]morpholine (also known as GDC 0941 and described in PCT Publication Nos. WO 09/036,082 and WO 09/055,730), 2-Methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in PCT Publication No. WO 06/122806), LY294002 (2-(4-Morpholinyl)-8-phenyl-4H-1-benzopyran-4-one available from Axon Medchem), PI 103 hydrochloride (3-[4-(4-morpholinylpyrido-[3′,2′: 4,5]furo[3,2-d]pyrimidin-2-yl]phenol hydrochloride available from Axon Medchem), PIK 75 (N′—[(IE)-(6-bromoinddazo[1,2-a]pyridin-3-yl)methylene]-N,2-dimethyl-5-nitrobenzenesulfono-hydrazide hydrochloride available from Axon Medchem), PIK 90 (N-(7,8-dimethoxy-2,3-dihydro-imidazo[1,2-c]quinazolin-5-yl)-nicotinamide available from Axon Medchem), GDC-0941 bismesylate (2-(1H-Indazol-4-yl)-6-(4-methanesulfonyl-piperazin-1-ylmethyl)-4-mo holin-4-yl-thieno[3,2-d]pyrimidine bismesylate available from Axon Medchem), AS-252424 (5-[1-[5-(4-fluoro-2-hydroxy-phenyl)-furan-2-yl]-meth-(Z)-ylidene]-thiazolidine-2,4-dione available from Axon Medchem), and TGX-221 (7-Methyl-2-(4-morpholinyl)-9-[1-(phenylamino)ethyl]-4H-pyrido-[1,2-a]pyrirnidin-4-one available from Axon Medchem), XL-765, and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136.

AKT inhibitors include, but are not limited to, Akt-1-1 (inhibits Aktl) (Barnett et al. (2005) Biochem. J., 385 (Pt. 2), 399-408); Akt-1-1,2 (Barnett et al. (2005) Biochem. J. 385 (Pt. 2), 399-408); API-59CJ-Ome (e.g., Jin et al. (2004) Br. J. Cancer 91, 1808-12); 1-H-imidazo[4,5-c]pyridinyl compounds (e.g., WO05011700); indole-3-carbinol and derivatives thereof (e.g., U.S. Pat. No. 6,656,963; Sarkar and Li (2004) J Nutr. 134 (12 Suppl), 3493S-3498S); perifosine; Dasmahapatra et al. (2004) Clin. Cancer Res. 10 (15), 5242-52, 2004); phosphatidylinositol ether lipid analogues (e.g., Gills and Dennis (2004) Expert. Opin. Investig. Drugs 13, 787-97); and triciribine (TCN or API-2 or NCI identifier: NSC 154020; Yang et al. (2004) Cancer Res. 64, 4394-9).

TOR inhibitors include, but are not limited to, inhibitors include AP-23573, CCI-779, everolimus, RAD-001, rapamycin, temsirolimus, ATP-competitive TORC1/TORC2 inhibitors, including PI-103, PP242, PP30 and Torin 1. Other TOR inhibitors in FKBP12 enhancer; rapamycins and derivatives thereof, including: CCI-779 (temsirolimus), RAD001 (Everolimus; WO 9409010) and AP23573; rapalogs, e.g. as disclosed in WO 98/02441 and WO 01/14387, e.g. AP23573, AP23464, or AP23841; 40-(2-hydroxyethyl)rapamycin, 40-[3-hydroxy (hydroxymethyl)methylpropanoate]-rapamycin, 40-epi-(tetrazolyt)-rapamycin (also called ABT578), 32-deoxorapamycin, 16-pentynyloxy-32(S)-dihydrorapanycin, and other derivatives disclosed in WO 05005434; derivatives disclosed in U.S. Pat. No. 5,258,389, WO 94/090101, WO 92/05179, U.S. Pat. Nos. 5,118,677, 5,118,678, 5,100,883, 5,151,413, 5,120,842, WO 93/111130, WO 94/02136, WO 94/02485, WO 95/14023, WO 94/02136, WO 95/16691, WO 96/41807, WO 96/41807 and U.S. Pat. No. 5,256,790; and phosphorus-containing rapamycin derivatives (e.g., WO 05016252). MC1-1 inhibitors include, but are not limited to, AMG-176, MIK665, and S63845. Proteasome inhibitors include, but are not limited to, Kyprolis® (carfilzomib), Velcade® (bortezomib), and oprozomib.

Immune therapies include, but are not limited to, anti-PD-1 agents, anti-PD-L1 agents, anti-CTLA-4 agents, anti-LAG1 agents, and anti-OX40 agents.

Monoclonal antibodies include, but are not limited to, Darzalex® (daratumumab), Herceptin® (trastuzumab), Avastin® (bevacizumab), Rituxan® (rituximab), Lucentis® (ranibizumab), and Eylea® (aflibercept).

In some embodiments, the compounds of Formula (I) are used in combination with an anti-CTLA-4 antibody, e.g., ipilumumab.

The invention further relates to a method of treating cancer in a human patient comprising administration of a compound of the invention (i.e., a compound of Formula (I)) and a PD-1 antagonist to the patient. The compound of the invention and the PD-1 antagonist may be administered concurrently or sequentially.

In particular embodiments, the PD-1 antagonist is an anti-PD-1 antibody, or antigen binding fragment thereof. In alternative embodiments, the PD-1 antagonist is an anti-PD-L1 antibody, or antigen binding fragment thereof. In some embodiments, the PD-1 antagonist is pembrolizumab (KEYTRUDA™, Merck & Co., Inc., Kenilworth, NJ, USA), nivolumab (OPDIVO™, Bristol-Myers Squibb Company, Princeton, NJ, USA), cemiplimab (LIBTAYO™, Regeneron Pharmaceuticals, Inc., Tarrytown, NY, USA), atezolizumab (TECENTRIQ™, Genentech, San Francisco, CA, USA), durvalumab (IMFINZI™, AstraZeneca Pharmaceuticals LP, Wilmington, DE), or avelumab (BAVENCIO™, Merck KGaA, Darmstadt, Germany).

In some embodiments, the PD-1 antagonist is pembrolizumab. In particular sub-embodiments, the method comprises administering 200 mg of pembrolizumab to the patient about every three weeks. In other sub-embodiments, the method comprises administering 400 mg of pembrolizumab to the patient about every six weeks.

In further sub-embodiments, the method comprises administering 2 mg/kg of pembrolizumab to the patient about every three weeks. In particular sub-embodiments, the patient is a pediatric patient.

In some embodiments, the PD-1 antagonist is nivolumab. In particular sub-embodiments, the method comprises administering 240 mg of nivolumab to the patient about every two weeks. In other sub-embodiments, the method comprises administering 480 mg of nivolumab to the patient about every four weeks.

In some embodiments, the PD-1 antagonist is cemiplimab. In particular embodiments, the method comprises administering 350 mg of cemiplimab to the patient about every 3 weeks.

In some embodiments, the PD-1 antagonist is atezolizumab. In particular sub-embodiments, the method comprises administering 1200 mg of atezolizumab to the patient about every three weeks.

In some embodiments, the PD-1 antagonist is durvalumab. In particular sub-embodiments, the method comprises administering 10 mg/kg of durvalumab to the patient about every two weeks.

In some embodiments, the PD-1 antagonist is avelumab. In particular sub-embodiments, the method comprises administering 800 mg of avelumab to the patient about every two weeks.

The compounds of the invention can be used in combination with the agents disclosed herein or other suitable agents, depending on the condition being treated. Hence, in some embodiments the one or more compounds of the invention will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound of Formula (I) and any of the agents described above can be formulated together in the same dosage form and administered simultaneously. Alternatively, a compound of Formula (I) and any of the agents described above can be simultaneously administered, wherein both the agents are present in separate formulations. In another alternative, a compound of Formula (I) can be administered just followed by and any of the agents described above, or vice versa. In some embodiments of the separate administration protocol, a compound of Formula (I) and any of the agents described above are administered a few minutes apart, or a few hours apart, or a few days apart.

As one aspect of the present invention contemplates the treatment of the disease/conditions with a combination of pharmaceutically active compounds that may be administered separately, the invention further relates to combining separate pharmaceutical compositions in kit form. The kit comprises two separate pharmaceutical compositions: a compound of Formula (I), and a second pharmaceutical compound. The kit comprises a container for containing the separate compositions such as a divided bottle or a divided foil packet. Additional examples of containers include syringes, boxes, and bags. In some embodiments, the kit comprises directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans. The terms “administration of” and or “administering a” compound should be understood to mean providing a compound of the invention or a prodrug of a compound of the invention to the individual in need of treatment.

The term “composition” as used herein is intended to encompass a product comprising specified ingredients in predetermined amounts or proportions, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions of the present invention encompass any composition made by mixing a compound of the present invention and a pharmaceutically acceptable carrier.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients that are suitable for the manufacture of tablets. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredients are mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Aqueous suspensions, oily suspensions, dispersible powders or granules, oil-in-water emulsions, and sterile injectable aqueous or oleagenous suspension may be prepared by standard methods known in the art. By “pharmaceutically acceptable” it is meant the carrier, diluent or excipient must be compatible with the other ingredients of the formulation and not deleterious to the recipient thereof.

The subject compounds are further useful in a method for the prevention, treatment, control, amelioration, or reduction of risk of the diseases, disorders and conditions noted herein. The dosage of active ingredient in the compositions of this invention may be varied, however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease, the patient's weight, special diets then being followed by a patient, concurrent medication, and other factors which those skilled in the art will recognize. Generally, dosage levels of between 0.001 to 10 mg/kg of body weight daily are administered to the patient, e.g., humans and elderly humans. The dosage range will generally be about 0.5 mg to 1.0 g per patient per day which may be administered in single or multiple doses. In one embodiment, the dosage range will be about 0.5 mg to 500 mg per patient per day; in another embodiment about 0.5 mg to 200 mg per patient per day; and in yet another embodiment about 5 mg to 50 mg per patient per day. Pharmaceutical compositions of the present invention may be provided in a solid dosage formulation such as comprising about 0.5 mg to 500 mg active ingredient, or comprising about 1 mg to 250 mg active ingredient. The pharmaceutical composition may be provided in a solid dosage formulation comprising about 1 mg, 5 mg, 10 mg, 25 mg, 50 mg, 100 mg, 200 mg or 250 mg active ingredient. For oral administration, the compositions may be provided in the form of tablets containing 1.0 to 1000 milligrams of the active ingredient, such as 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, such as once or twice per day.

While preferred embodiments of the present invention have been shown and described herein, it will be obvious to those skilled in the art that such embodiments are provided by way of example only. Numerous variations, changes and substitutions will now occur to those skilled in the art without departing from the invention. It should be understood that various alternatives to the embodiments of the invention described herein may be employed in practicing the invention. It is intended that the appended claims define the scope of the invention and that methods and structures within the scope of these claims and their equivalents be covered thereby.

Starting materials and the requisite intermediates are in some cases commercially available, or can be prepared according to literature procedures or as illustrated herein. The compounds of this invention may be prepared by employing reactions as shown in the following schemes, in addition to other standard manipulations that are known in the literature or exemplified in the experimental procedures. Substituent numbering as shown in the schemes does not necessarily correlate to that used in the claims and often, for clarity, a single substituent is shown attached to the compound where multiple substituents are allowed under the definitions hereinabove. Reactions used to generate the compounds of this invention are prepared by employing conditions as shown in the schemes and examples herein, as well as using other standard manipulations such as ester hydrolysis, cleavage of protecting groups, etc., as may be known in the literature or exemplified in the experimental procedures. Starting materials are made according to procedures known in the art or as illustrated herein.

In some cases the final product may be further modified, for example, by manipulation of substituents. These manipulations may include, but are not limited to, reduction, oxidation, alkylation, acylation, and hydrolysis reactions which are commonly known to those skilled in the art. In some cases the order of carrying out the foregoing reaction schemes may be varied to facilitate the reaction or to avoid unwanted reaction products. The following examples are provided so that the invention might be more fully understood.

The representative examples of the compounds of the invention are illustrated in the following non-limiting schemes and Examples.

### General

Starting materials used were obtained from commercial sources or prepared in other examples, unless otherwisely noted. The progress of reactions was often monitored by TLC or LC-MS. The LC-MS was recorded using one of the following methods. Preparative thin layer chromatography (PTLC) separations described herein were typically performed on 20×20 cm plates (500 micron thick silica gel).

Chromatographic purifications were typically performed using Biotage® Isolera One automated systems running Biotage® Isolera One 2.0.6 software (Biotage LLC, Charlotte, NC USA). Flow rates were the default values specified for the particular column in use. Reverse phase chromatography was performed using elution gradients of water and acetonitrile on Biotage® KP-C18-HS Flash+ columns (Biotage LLC) of various sizes. Typical loading was between 1:50 and 1:1000 crude sample: RP SiO₂ by weight. Normal phase chromatography was performed using elution gradients of various solvents (e.g. hexane, ethyl acetate, methylene chloride, methanol, acetone, chloroform, MTBE, etc.). The columns were Biotage® SNAP Cartridges containing KP-SIL or Biotage® SNAP Ultra (25 μm spherical particles) of various sizes (Biotage LLC). Typical loading was between 1:10 to 1:150 crude sample: SiO₂ by weight. Alternatively, silica gel chromatography was performed on a Biotage Horizon flash chromatography system.

¹H NMR analyses of intermediates and exemplified compounds were typically performed on an Agilent Technologies 400/54 (Agilent Technologies, Santa Clara, CA, US) or Bruker® Ascend™ 400 spectrometer (Bruker BioSpin AG, Faellanden Switzerland) (operating at 400 MHz) at 298° K following standard operating procedure suggested by manufacturer. Reference frequency was set using TMS as an internal standard. Typical deuterated solvents were utilized as indicated in the individual examples.

LCMS (Liquid Chromatography-Mass Spectrometry) analyses were typically performed using one of the two conditions listed below:
(1) LCMS spectra were taken on an Agilent Technologies 1260 Infinity coupled to 6120 Quadrupole spectrometer. The mobile phase for the LC was acetontrile (A) and water (B) with 0.01% formic acid, and the eluent gradient was from 5-95% A in 6.0 min, 60-95% A in 5.0 min, 80-100% A in 5.0 min and 85-100% A in 10 min using a SBC18 50 mm×4.6 mm×2.7 μm capillary column. Mass spectra (MS) were measured by electrospray ion-mass spectroscopy (ESI). All temperatures are in degrees Celsius unless otherwise noted; and
(2) LCMS analysis of intermediates and exemplified compounds was performed on an Agilent Technologies 1200 Series HPLC system coupled to an Agilent Technologies 6150 Quadrapole LC/MS detector. Analytes were detected by UV absorbance at 220 and 254 nm. Analyte ions were detected by mass spectrometry in both negative and positive modes (110-800 amu scan range, API-ES ionization). A long HPLC method was run on a Phenomenex R Kinetex 2.6μ C18 100 Å, 30×3.00 mm column (Phenomenex, Torrance, CA). The column temperature was set at 40° C. UV absorptions were detected at 220 and 254 nm. Samples were prepared as a solution in about 1:1 (v/v) acetonitrile:water mixture. Flow rate was about 0.80 mL/minute. Elution solvents were acetonitrile and water each containing 0.1% formic acid. In a typical run, a linear gradient starting with 5% acetonitrile and 95% water and ending with 95% acetonitrile and 5% water over 12 minutes was carried out. At the end of each run, the column was washed with 95% acetonitrile and 5% water for 2 minutes.

Typically, analytical HPLC mass spectrometry conditions were as follows:
1) LC1: Column: SB—C18 50 mm×4.6 mm×2.7 μm; Temperature: 50° C.; Eluent: 5:95 v/v acetonitrile/water+0.01% formic acid in 6 min; Flow Rate: 1.5 mL/min; Injection 5 μL; Detection:PDA, 200-600 nm; MS: mass range 150-750 amu; positive ion electrospray ionization;
2) LC2: Column: SB—C18 50 mm×4.6 mmx 2.7 μm; Temperature: 50° C.; Eluent: 5:95 to 95:5 v/v acetonitrile/water+0.05% TFA over 3.00 min; Flow Rate: 1.5 mL/min; Injection 5 μL; Detection: PDA, 200-600 nm; MS: mass range 150-750 amu; positive ion electrospray ionization; and
3) LC3: Column: SB—C18 50 mm×4.6 mm×2.7 μm; Temperature: 50° C.; Eluent: 10:90 to 98:2 v acetonitrile/water+0.05% TFA over 3.75 min; Flow Rate: 1.0 mL/min; Injection 10 μL; Detection: PDA, 200-600 nm; MS: mass range 150-750 amu; positive ion electrospray ionization.

Preparative HPLC were carried out with one of the two conditions listed below:
1) Condition 1: GILSON Preparative HPLC System (Gilson Incorported, Middleton, WI, USA); Column: SHISEIDO CAPCELL PAK, MG (Shiseido Co., Ltd., Japan); C18, 20 mm×250 mm, 5 μm; Mobile phase: Water+0.1% trifluoroacetic acid; ACN+0.1% trifluoroacetic acid; Method: 15 minutes gradient elution; Initial organic: 10%; Final organic: 80%; UV1:240; UV2:230; Flow: 15 ml/min.
2) Condition 2: GILSON Preparative HPLC System; Column: SunFire® Prep C18 OBD 5 μm, 19 mm×150 mm (Waters Corporation, Milford, MA USA); Mobile phase: Water+0.1% trifluoroacetic acid; ACN+0.1% trifluoroacetic acid; Method: 20 minutes gradient elution; Initial organic: 10%; Final organic: 80%; UV1:220; UV2:254; Flow: 15 ml/min.
3) Condition 3: GILSON Preparative HPLC System, GX-281, Column: Welch ultimate XB—C18, 21.2 mm×250 mm, 5 μm (Welch Materials, Inc., West Haven, CT USA), MeCN/H₂O with 0.05% TFA or 0.1% HCOOH.

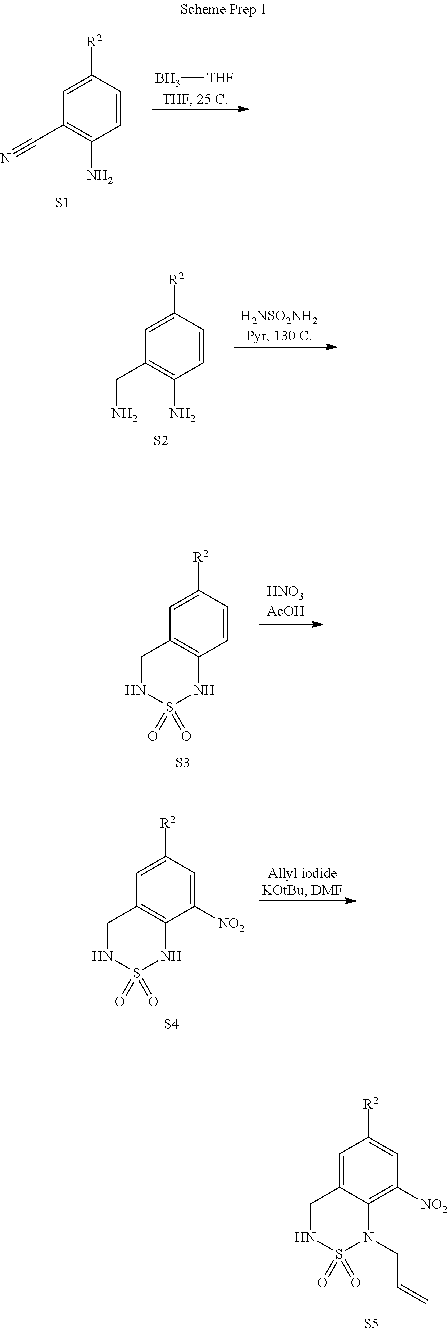

Scheme Prep 1 illustrates a synthetic sequence for the preparation of cyclic sulfamide derivatives such as S5 from ortho cyano aniline derivatives such as S1, and S4, S5 can serve as useful intermediates in the preparation of myriad tricyclic sulfamide compounds. The sequence starts from ortho cyano aniline S1 or diamine S2. If the the sequence begins with ortho cyano aniline S1, reduction to diamine S2 is facilitated by treatment with borane tetrahydrofuran complex. Formation of the cyclic sulfamide S3 is achieved by heating diamine S2 with an excess of sulfamide in pyridine. Nitration of S3 in a mixture of acetic acid and nitric acid affords the ortho nitro aniline derivative S4. Allylation of the aniline nitrogen in ortho nitro aniline derivative S4 to afford allyl protected S5 is accomplished by pre-treatment with stoichiometric potassium t-butoxide followed by quenching with allyl iodide.

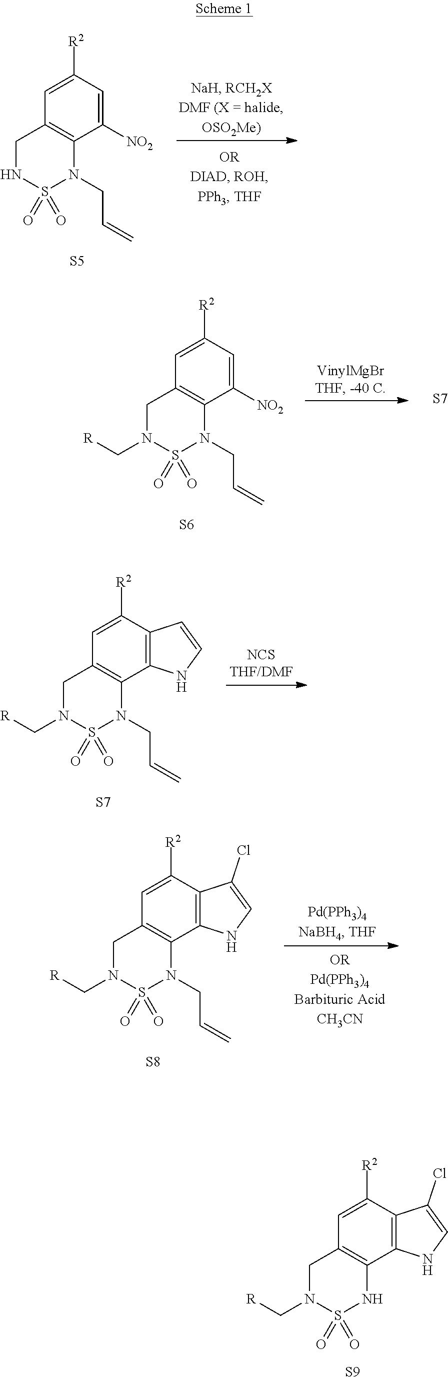

Scheme 1 illustrates a synthetic sequence for the preparation of tricyclic sulfamides such as S9 from cyclic sulfamides such as S5 as described in Scheme Prep 1. Sulfamide S5 is alkylated to afford functionalized sulfamide S6 by one of two possible methods: (1) treatment with an alkyl halide or pseudohalide in the presence of sodium hydride or (2) Mitsunobu reaction with the appropriate alcohol. Functionalized sulfamide S6 is cyclized to indole S7 by the Bartoli reaction following treatment with vinyl Grignard. Indole S7 is chlorinated with NCS to afford the chloroindole S8. The chloroindole S8 is deallylated to give tricyclic sulfamide S9 by one of two possible methods: (1) treatment with (tetrakis(triphenylphosphine) palladium (0)) and sodium borohydride or (2) treatment with (tetrakis(triphenylphosphine) palladium (0)) and barbituric acid.

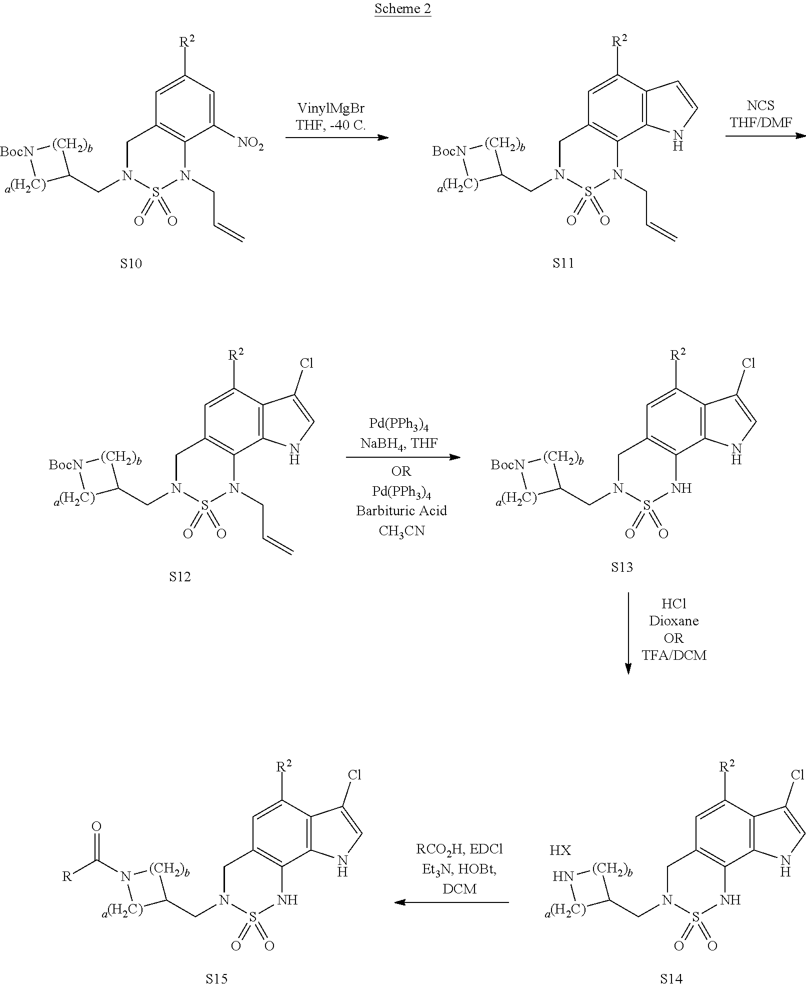

Scheme 2 illustrates a synthetic sequence for the synthesis of tricyclic sulfamides bearing an amide such as S15 from carbamates such as S10. Carbamate S10 is cyclized to indole S11 by Bartoli reaction following treatment with vinyl Grignard. Indole S11 is chlorinated with NCS to afford the chloroindole S12. Chloroindole S12 is deallylated to sulfamide S13 by one of two possible methods: (1) treatment with palladium tetrakis and sodium borohydride or (2) treatment with palladium tetrakis and barbituric acid. Sulfamide S13 loses its BOC group upon treatment with HCl or TFA to afford salt S14. Amide formation from salt S14 to afford tricyclic sulfamide S15 is achieved under standard peptide coupling conditions utilizing EDCl as the coupling agent.

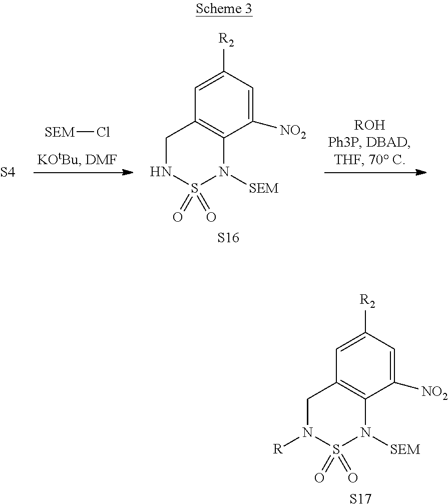

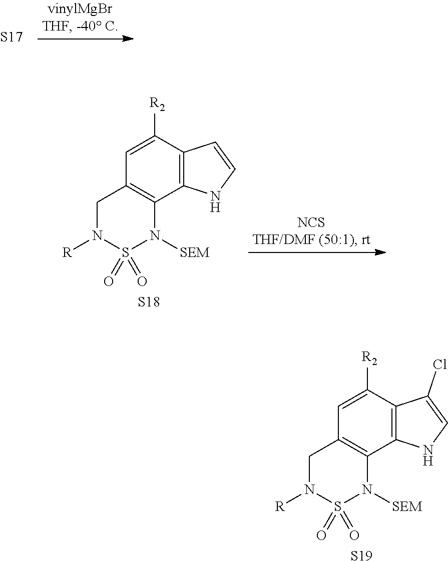

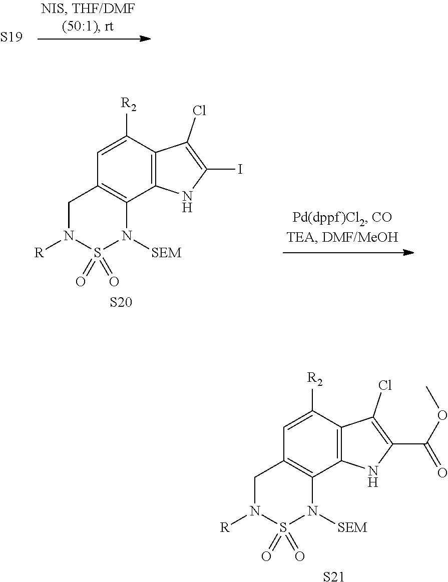

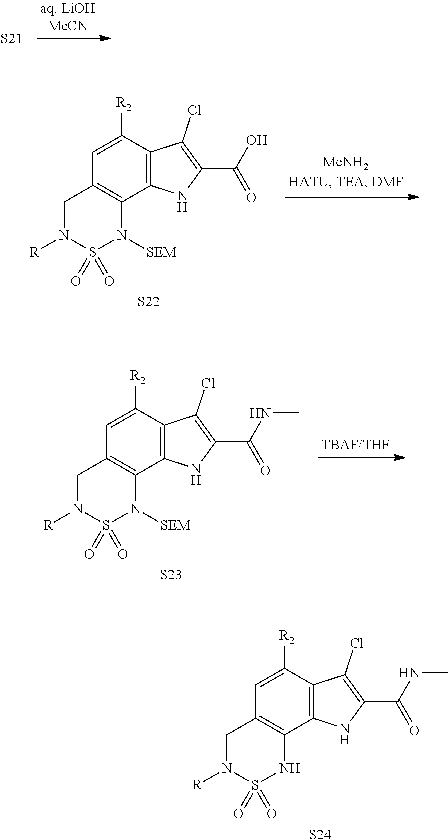

Scheme 3 illustrates a synthetic sequence for the preparation of tricyclic sulfamides bearing an amide substituent at 2-indole position such as S24 from nitro sulfamides such as S4 as shown in Scheme Prep 1. Protection of nitro sulfamide S4 with SEMCl followed with Mitsunobu reaction results in formation of alkylated nitro sulfamide S17. Nitro sulfamide S17 is cyclized to indole S18 by Bartoli reaction following treatment with vinyl Grignard. Indole S18 is chlorinated with NCS then iodinated with NIS to afford the chloro iodo indole S20. The iodo indole S20 is carbonylated by palladium-catalyzed cross-coupling reaction in presence of CO and methanol then hydrolyzed under basic condition to yield carboxylic acid substituted indole S22. Amide formation with methylamine followed by TBAF deprotection of SEM group affords tricyclic sulfamide S24.

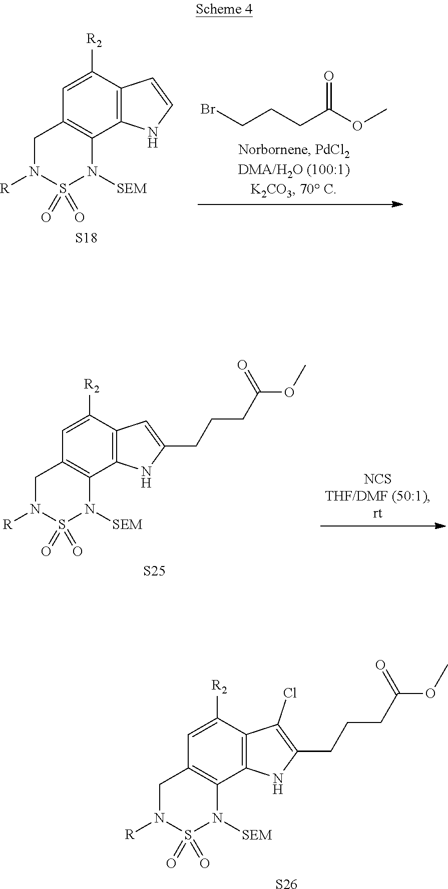

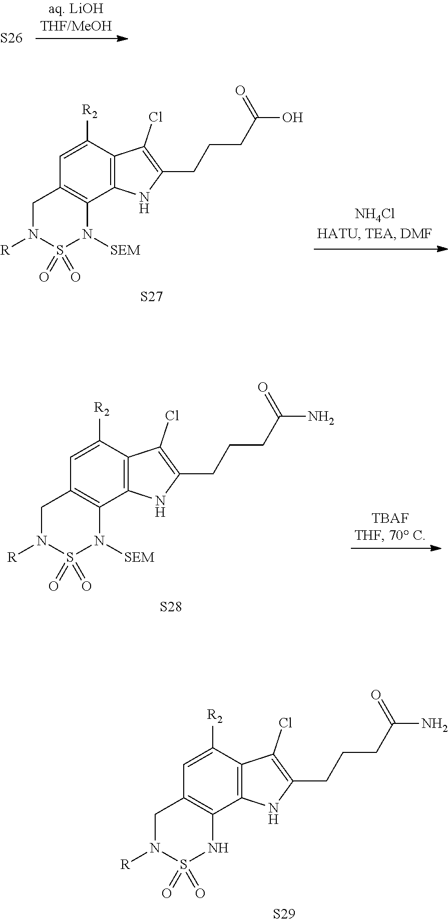

Scheme 4 illustrates a synthetic sequence for the preparation of 2-alkylated indole containing tricyclic sulfamides such as S29 from indole such as S18. Palladium-catalyzed direct 2-alkylation of indole S18 by norbornene mediated regioselective cascade C—H activation results in formation of 2-alkylated indole S25. Alkylated indole S25 is chlorinated with NCS treatment, followed by hydrolysis under basic condition to give 2-alkyl carboxylated indole S27. Amidation in the presence of ammonium chloride under amide formation conditions, followed by TBAF deprotection for SEM removal, generates 2-alkylated indole S29 containing tricyclic sulfamide.

### Preparatory Example 1

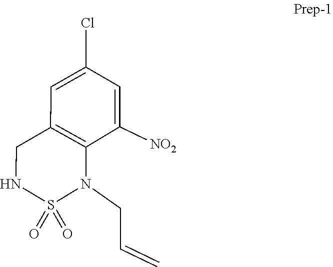 [SMILES: C=CCN1c2c(cc(Cl)cc2[N+](=O)[O-])CNS1(=O)=O]

### 1-allyl-6-chloro-8-nitro-3,4-dihydro-1H-benzo[c][1,2,6]thiadiazine 2,2-dioxide (Prep-1)

Preparatory Example 1 was prepared in an analogous manner to that outlined in Scheme Prep 1.

### Step 1. 2-(aminomethyl)-4-chloroaniline

To a solution of 2-amino-5-chlorobenzonitrile (20 g, 131.9 mmol) in dry THF (200 ml) at 0° C. under argon atmosphere was slowly added borane (158 ml, 158.3 mmol, ˜1.0 Min THF). The mixture was stirred at 0° C. for 10 min and 48 h at room temperature. The reaction mixture was cooled to 0° C., and quenched by the addition of HCl in MeOH (4 M). The precipitate was filtered and collected, and then treated with saturated aqueous ammonia solution. The resulting suspension was extracted with EtOAc (3×300 mL). The combined organic layers were dried over sodium sulfate. EtOAc was completely removed under reduced pressure to give 2-(aminomethyl)-4-chloroaniline as a solid. ESI MS [M-16]⁺ for C₇H₉ClN₂, calcd 140.05, found 140.1.

### Step 2. 6-chloro-3,4-dihydro-1H-benzo[c][1,2,6]thiadiazine 2,2-dioxide

To a stirred solution of 2-(aminomethyl)-4-chloroaniline (12.8 g, 81.73 mmol) in pyridine (130 mL) was added sulfuric diamide (23.5 g, 245.2 mmol) and the resulting mixture was heated to 130° C. for 5 h. After cooling, the reaction mixture was concentrated in vacuo. The residue was dissolved in DCM (200 ml) and the solution was washed with water (100 mL), dried over Na₂SO₄ and concentrated in vacuo. The resulting residue was crystallized from toluene to give 6-chloro-3,4-dihydro-1H-benzo[c][1,2,6]thiadiazine 2,2-dioxide as a solid. MS=216.9 (M−1).

### Step 3. 6-chloro-8-nitro-3,4-dihydro-1H-benzo[c][1,2,6]thiadiazine 2,2-dioxide

To a solution of 6-chloro-3,4-dihydro-1H-benzo[c][1,2,6]thiadiazine 2,2-dioxide (17 g, 77.75 mmol) in acetic acid (170 mL) was added fuming nitric acid (50 mL) dropwise at 0° C. After complete conversion of starting material, the mixture was concentrated in vacuo. The residue was poured into ice-water (100 mL) and treated with Na₂CO₃ to adjust pH to 6, The precipitate was filtered and dried to give 6-chloro-8-nitro-3,4-dihydro-1H-benzo[c][1,2,6]thiadiazine 2,2-dioxide as brown solid. MS=262.0 (M−1).

### Step 4. 1-allyl-6-chloro-8-nitro-3,4-dihydro-1H-benzo[c][1,2,6]thiadiazine 2,2-dioxide (Prep-1)

A solid mixture 6-chloro-8-nitro-3,4-dihydro-1H-benzo[c][1,2,6]thiadiazine 2,2-dioxide (24.8 g, 75.84 mmol) and potassium 2-methylpropan-2-olate (9.36 g, 83.44 mmol) was suspended in DMF (200 mL) and stirred at 25° C. for 15 min. The dark red solution was treated with allyl iodide (14.0 g, 83.44 mmol) and left to stir at room temperature until complete by LCMS. The reaction mixture was poured into 1000 ml of water and extracted with EtOAc (3×450 mL). The combined organics were washed with 400 mL of brine, dried over MgSO₄, filtered and concentrated to dryness. The residue was purified by column chromatography (silica gel, EtOAc/PE=1/1) to give 1-allyl-6-chloro-8-nitro-3,4-dihydro-1H-benzo[c][1,2,6]thiadiazine 2,2-dioxide (Prep-1). MS=302.0 (M−1).

### Example 1

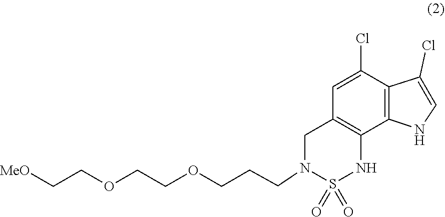 [SMILES: COCCOCCOCCCN1Cc2cc(Cl)c3c(Cl)c[nH]c3c2NS1(=O)=O]

### 6,7-dichloro-3-(3-(2-(2-methoxyethoxy)ethoxy)propyl)-1,3,4,9-tetrahydro-[1,2,6]thiadiazino[4,3-g]indole 2,2-dioxide (2)

Compounds 2 was prepared in an analogous manner to that outlined in Scheme 1.

### Step 1. 13-phenyl-2,5,8,12-tetraoxatridecane

The solution of 2-(2-methoxyethoxy)ethan-1-ol (500 mg, 4.16 mmol) in DMF (15 ml) was cooled to 0° C. NaH (150 mg, 6.24 mmol) (60%) was added. The mixture was stirred at 0° C. for 0.5 hr, and then was added ((3-bromopropoxy)methyl) benzene (1430 mg, 6.24 mmol). The resulting mixture was stirred at 20° C. for 18 hr under N₂. The mixture was evaporated under reduced pressure. The residue crude was purified by prep-HPLC (TFA) to give 13-phenyl-2,5,8,12-tetraoxatridecane (470 mg, 1.576 mmol). LCMS (ESI) m/z: 269.3 [M+H]⁺.

### Step 2. 3-(2-(2-methoxyethoxy)ethoxy)propan-1-ol

To a stirred solution of 13-phenyl-2,5,8,12-tetraoxatridecane (590 mg, 2.199 mmol) in MeOH (8 ml) was added Pd/C (46.8 mg, 0.440 mmol). The mixture was stirred at 20° C. for 18 h under H_{2 15} psi. LCMS showed starting material consumed. The mixture was filtered and concentrated to give 3-(2-(2-methoxyethoxy)ethoxy)propan-1-ol (450 mg, 2.020 mmol).

### Step 3. 1-allyl-6-chloro-3-(3-(2-(2-methoxyethoxy)ethoxy)propyl)-8-nitro-3,4-dihydro-1H-benzo[c][1,2,6]thiadiazine 2,2-dioxide

To the mixture of 1-allyl-6-chloro-8-nitro-3,4-dihydro-1H-benzo[c][1,2,6]thiadiazine 2,2-dioxide (Prep-1) (400 mg, 1.317 mmol) and 3-(2-(2-methoxyethoxy)ethoxy)propan-1-ol (265 mg, 1.97 mmol) in THF (10 ml) was added Ph3P (2764 mg, 10.54 mmol), followed by di-tert-butyl (E)-diazene-1,2-dicarboxylate (2426 mg, 10.54 mmol), then the reaction was stirred at 70° C. for 18 h. LCMS showed starting material consumed. After cooling down to room temperature, the mixture was concentrated under reduced pressure. The crude product was purified via reverse-phase chromatography (C18, Eluent of 0-30% H2O/MeCN gradient @ 30 mL/min) and silica column chromatography (SiO2, 30% EtOAc in ether) to give 1-allyl-6-chloro-3-(3-(2-(2-methoxyethoxy)ethoxy)propyl)-8-nitro-3,4-dihydro-1H-benzo[c][1,2,6]thiadiazine 2,2-dioxide (250 mg, 0.43 1 mmol). LC/MS (ESI) m/z: 420.2 [M+H]⁺.

### Step 4. 1-allyl-6-chloro-3-(3-(2-(2-methoxyethoxy)ethoxy)propyl)-1,3,4,9-tetrahydro-[1,2,6]thiadiazino[4,3-g]indole 2,2-dioxide

To the solution of 1-allyl-6-chloro-3-(3-(2-(2-methoxyethoxy)ethoxy)propyl)-8-nitro-3,4-dihydro-1H-benzo[c][1,2,6]thiadiazine 2,2-dioxide (40 mg, 0.086 mmol) in THF was added vinylmagnesium bromide (377 μl, 0.604 mmol) at −78° C., then the mixture was stirred −40° C. for 1 h. TLC showed starting material consumed. The mixture was quenched with sat. NH4Cl solution (2 mL), and extracted with EtOAc (10 mL×2), the organic phase was dried over Na2SO4, filtered and concentrated, purified by p-TLC (ether/EtOAc=2/1) to give 1-allyl-6-chloro-3-(3-(2-(2-methoxyethoxy)ethoxy)propyl)-1,3,4,9-tetrahydro-[1,2,6]thiadiazino[4,3-g]indole 2,2-dioxide (10 mg, 0.020 mmol).

### Step 5. 1-allyl-6,7-dichloro-3-(3-(2-(2-methoxyethoxy)ethoxy)propyl)-1,3,4,9-tetrahydro-[1,2,6]thiadiazino[4,3-g]indole 2,2-dioxide

The mixture of 1-allyl-6-chloro-3-(3-(2-(2-methoxyethoxy)ethoxy)propyl)-1,3,4,9-tetrahydro-[1,2,6]thiadiazino[4,3-g]indole 2,2-dioxide (10 mg, 0.022 mmol) and NCS (4.37 mg, 0.033 mmol) in THF (1 ml) and DMF (0.1 ml) was stirred at 20° C. for 17 h. LCMS showed most starting material consumed. The mixture was diluted with 2 mL of EtOAc, added sat. Na2S2O3 (0.1 mL), dried over Na2SO4, filtered and concentrated to give the crude 1-allyl-6,7-dichloro-3-(3-(2-(2-methoxyethoxy)ethoxy)propyl)-1,3,4,9-tetrahydro-[1,2,6]thiadiazino[4,3-g]indole 2,2-dioxide (12 mg, 0.015 mmol), which was used in next step without further purification. LCMS (ESI) m/z: 492.1 [M+H]+.

### Step 6. 6,7-dichloro-3-(3-(2-(2-methoxyethoxy)ethoxy)propyl)-1,3,4,9-tetrahydro-[1,2,6]thiadiazino[4,3-g]indole 2,2-dioxide

To the mixture of 1-allyl-6,7-dichloro-3-(3-(2-(2-methoxyethoxy)ethoxy)propyl)-1,3,4,9-tetrahydro-[1,2,6]thiadiazino[4,3-g]indole 2,2-dioxide (12 mg, 0.024 mmol) and Pd(Ph3P) 4 (14.08 mg, 0.012 mmol) in THF (2 ml) was added NaBH4 (1.844 mg, 0.049 mmol), then the mixture was stirred at 20° C. for 17 h. LCMS showed starting material consumed and desired product formed. The mixture was purified by prep-HPLC to give the compound 6,7-dichloro-3-(3-(2-(2-methoxyethoxy)ethoxy)propyl)-1,3,4,9-tetrahydro-[1,2,6]thiadiazino[4,3-g]indole 2,2-dioxide (2.82 mg, 5.86 μmol). LCMS (ESI) m/z: 451.9 [M+H]+.

The following compounds as shown in Table 1 were prepared using procedures analogous to those described in Example 1 and shown in Scheme 1 using the appropriate starting materials.

TABLE 1

| | | | Exact Mass |
|---|---|---|---|
| Compd. | | | [M + H]⁺ or |
| No. | Structure | Name | [M − H]⁻ |
| 1 | 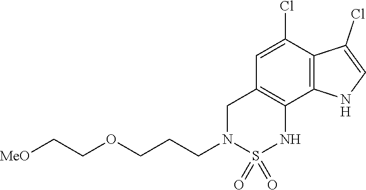 [SMILES: COCCOCCCN1Cc2cc(Cl)c3c(Cl)c[nH]c3c2NS1(=O)=O] | 6,7-dichloro-3-(3-(2- methoxyethoxy)propyl)- 1,3,4,9-tetrahydro- [1,2,6]thiadiazino[4,3-g]indole 2,2-dioxide | Calc'd 408.05, found 408.0 |
| 2 | 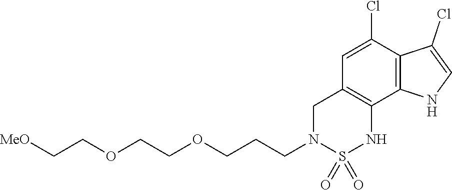 [SMILES: COCCOCCOCCCN1Cc2cc(Cl)c3c(Cl)c[nH]c3c2NS1(=O)=O] | 6,7-dichloro-3-(3-(2-(2- methoxyethoxy)ethoxy)propyl)- 1,3,4,9-tetrahydro- [1,2,6]thiadiazino[4,3-g]indole 2,2-dioxide | Calc'd 452.07, found 451.9 |
| 3 | 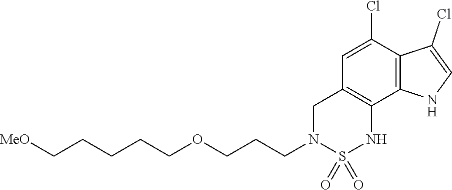 [SMILES: COCCCCCOCCCN1Cc2cc(Cl)c3c(Cl)c[nH]c3c2NS1(=O)=O] | 6,7-dichloro-3-(3-((5- methoxypentyl)oxy)propyl)- 1,3,4,9-tetrahydro- [1,2,6]thiadiazino[4,3-g]indole 2,2-dioxide | Calc'd 450.09, found 450.1 |
| 4 | 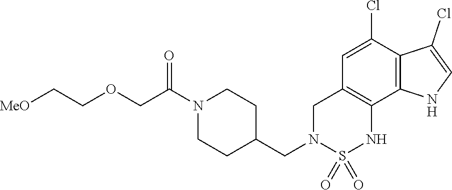 [SMILES: COCCOCC(=O)N1CCC(CN2Cc3cc(Cl)c4c(Cl)c[nH]c4c3NS2(=O)=O)CC1] | 1-(4-((6,7-dichloro-2,2-dioxido- 4,9-dihydro- [1,2,6]thiadiazino[4,3-g]indol- 3(1H)-yl)methyl)piperidin-1- yl)-2-(2-methoxyethoxy)ethan- 1-one | Calc'd 505.10, found 505.1 |
| 5 | 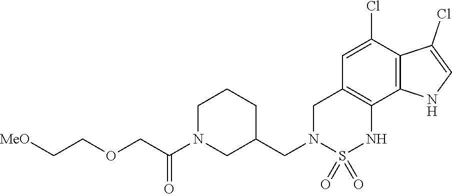 [SMILES: COCCOCC(=O)N1CCCC(CN2Cc3cc(Cl)c4c(Cl)c[nH]c4c3NS2(=O)=O)C1] | 1-(3-((6,7-dichloro-2,2-dioxido- 4,9-dihydro- [1,2,6]thiadiazino[4,3-g]indol- 3(1H)-yl)methyl)piperidin-1- yl)-2-(2-methoxyethoxy)ethan- 1-one | Calc'd 505.10, found 505.1 |

### Example 2

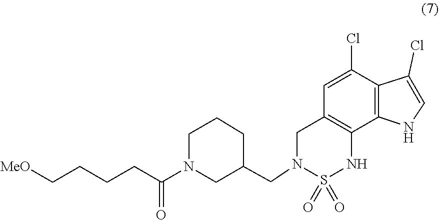 [SMILES: COCCCCC(=O)N1CCCC(CN2Cc3cc(Cl)c4c(Cl)c[nH]c4c3NS2(=O)=O)C1]

### 1-(3-((6,7-dichloro-2,2-dioxido-4,9-dihydro-[1,2,6]thiadiazino[4,3-g]indol-3 (1H)-yl)methyl) piperidin-1-yl)-5-methoxypentan-1-one (7)

Compound 7 was prepared in an analogous manner to that outlined in Scheme 2.

### Step 1, tert-butyl 3-((1-allyl-6-chloro-8-nitro-2,2-dioxido-1,4-dihydro-3H-benzo[c][1,2,6]thiadiazin-3-yl)methyl) piperidine-1-carboxylate

To the mixture of 1-allyl-6-chloro-8-nitro-3,4-dihydro-1H-benzo[c][1,2,6]thiadiazine 2,2-dioxide (Prep-1) (1 g, 3.29 mmol) and tert-butyl 3-(hydroxymethyl) piperidine-1-carboxylate (1.06 g, 4.94 mmol) in THF (10 ml) was added Ph3P (4.32 g, 16.46 mmol), followed by di-tert-butyl (E)-diazene-1,2-dicarboxylate (3.79 g, 16.46 mmol), then the reaction was stirred at 70° C. for 18 h. LCMS showed starting material consumed and desired product formed. After cooling down to room temperature, the mixture was concentrated under reduced pressure. The crude product was purified by silica column chromatography (SiO2, 30% EtOAc in pentane ether) to give tert-butyl 3-((1-allyl-6-chloro-8-nitro-2,2-dioxido-1,4-dihydro-3H-benzo[c][1,2,6]thiadiazin-3-yl)methyl) piperidine-1-carboxylate (1.56 g, 2.80 mmol). LC/MS: MS (ESI) m/z: 523.2 [M+Na+].

### Step 2, tert-butyl 3-((1-allyl-6-chloro-2,2-dioxido-4,9-dihydro-[1,2,6]thiadiazino[4,3-g]indol-3 (1H)-yl)methyl) piperidine-1-carboxylate

To a stirred solution of tert-butyl 3-((1-allyl-6-chloro-8-nitro-2,2-dioxido-1,4-dihydro-3H-benzo[c][1,2,6]thiadiazin-3-yl)methyl) piperidine-1-carboxylate (800 mg, 1.597 mmol) in THF (10 ml) was added vinylmagnesium bromide (5.99 ml, 9.58 mmol) [1.6M in 2Me-THF] dropwise at −78° C. The mixture was allowed to warm up to −40° C., and stirred for further 3 h. LCMS showed starting material consumed and desired product formed. The mixture was quenched with sat. NH4Cl solution (5 mL) and warmed up to room temperature then extrated with EtOAc (15 mL). The organic phase was concentrated under reduced pressure and then purified pre-TLC (ether/EtOAc=5:1) to give tert-butyl 3-((1-allyl-6-chloro-2,2-dioxido-4,9-dihydro-[1,2,6]thiadiazino[4,3-g]indol-3 (1H)-yl)methyl) piperidine-1-carboxylate (220 mg, 0.356 mmol). LC/MS: MS (ESI) m/z: 495.2 [M-100+H+].

### Step 3, tert-butyl 3-((1-allyl-6,7-dichloro-2,2-dioxido-4,9-dihydro-[1,2,6]thiadiazino[4,3-g]indol-3 (1H)-yl)methyl) piperidine-1-carboxylate

To the solution of tert-butyl 3-((1-allyl-6-chloro-2,2-dioxido-4,9-dihydro-[1,2,6]thiadiazino[4,3-g]indol-3 (1H)-yl)methyl) piperidine-1-carboxylate (70 mg, 0.141 mmol) in THF (2 ml)/DMF (0.04 ml) was added NCS (16.99 mg, 0.127 mmol), then the mixture was stirred 25° C. for 17 h. LCMS showed starting material consumed. The mixture was diluted with EtOAc (2 mL), washed with water (1 mL) and brine (1 mL), dried over Na2SO4, filtered and concentrated to give the crude tert-butyl 3-((1-allyl-6,7-dichloro-2,2-dioxido-4,9-dihydro-25 [1,2,6]thiadiazino[4,3-g]indol-3 (1H)-yl)methyl) piperidine-1-carboxylate (60 mg, 0.091 mmol), which was used in next step without further purification. LC/MS: MS (ESI) m/z: 529.2 [M+H+].

### Step 4. 1-allyl-6,7-dichloro-3-(piperidin-3-ylmethyl)-1,3,4,9-tetrahydro-[1,2,6]thiadiazino[4,3-g]indole 2,2-dioxide

To the solution of tert-butyl 3-((1-allyl-6,7-dichloro-2,2-dioxido-4,9-dihydro-[1,2,6]thiadiazino[4,3-g]indol-3 (1H)-yl)methyl) piperidine-1-carboxylate (45 mg, 0.085 mmol) in DCM (0.02 ml) was added TFA (1 ml), then the mixture was stirred 25° C. for 1 h. LCMS showed starting material consumed. The mixture was concentrated to give the 1-allyl-6,7-dichloro-3-(piperidin-3-ylmethyl)-1,3,4,9-tetrahydro-[1,2,6]thiadiazino[4,3-g]indole 2,2-dioxide TFA salt (50 mg, 0.083 mmol). LC/MS: MS (ESI) m/z: 429.1 [M+H+].

### Step 5. 1-(3-((1-allyl-6,7-dichloro-2,2-dioxido-4,9-dihydro-[1,2,6]thiadiazino[4,3-g]indol-3 (1H)-yl)methyl) piperidin-1-yl)-5-methoxypentan-1-one

To a stirred solution of 5-methoxypentanoic acid (36.5 mg, 0.276 mmol) in DCM (1 ml) was added 1-propanephosphonic anhydride (110 mg, 0.138 mmol), DIEA (0.048 ml, 0.276 mmol) and 1-allyl-6,7-dichloro-3-(piperidin-3-ylmethyl)-1,3,4,9-tetrahydro-[1,2,6]thiadiazino[4,3-g]indole 2,2-dioxide TFA salt (50 mg, 0.092 mmol). The mixture was stirred at 25° C. for 18 h. LCMS showed starting material consumed and desired product formed. The mixture was washed with water (5 ml), then extracted with DCM (5 ml×3). The organic phase was washed with brine (5 ml), dried over Na2SO4, filtered, concentrated, then purified by pre-HPLC (TFA) to give 1-(3-((1-allyl-6,7-dichloro-2,2-dioxido-4,9-dihydro-[1,2,6]thiadiazino[4,3-g]indol-3 (1H)-yl)methyl) piperidin-1-yl)-5-methoxypentan-1-one (30 mg, 0.050 mmol). LC/MS: MS (ESI) m/z: 543.2 [M+H+].

### Step 6. 1-(3-((6,7-dichloro-2,2-dioxido-4,9-dihydro-[1,2,6]thiadiazino[4,3-g]indol-3 (1H)-yl)methyl) piperidin-1-yl)-5-methoxypentan-1-one

To a solution of 1-(3-((1-allyl-6,7-dichloro-2,2-dioxido-4,9-dihydro-[1,2,6]thiadiazino[4,3-g]indol-3 (1H)-yl)methyl) piperidin-1-yl)-5-methoxypentan-1-one (30 mg, 0.055 mmol) and tetrakis)triphenylphosphine) palladium (0) (6.38 mg, 5.52 μmol) in THF (1 ml), was added NaBH4 (4.18 mg, 0.110 mmol). The mixture was stirred at 25° C. for 18 hr. LCMS showed desired product formed. The mixture was concentrated then purified by pre-HPLC (TFA) to give 1-(3-((6,7-dichloro-2,2-dioxido-4,9-dihydro-[1,2,6]thiadiazino[4,3-g]indol-3 (1H)-yl)methyl) piperidin-1-yl)-5-methoxypentan-1-one (7). ¹H NMR (400 MHZ, DMSO-d6) δ ppm 1.23 (brs, 4H) 1.50 (br s, 4H) 1.73 (br s, 2H) 1.93-2.11 (m, 1H) 2.29 (br s, 2H) 2.73 (brd, J=6.26 Hz, 2H) 2.80-3.06 (m, 2H) 3.20 (br d, J=5.87 Hz, 3H) 3.65-3.79 (m, 1H) 3.94-4.33 (m, 1H) 4.62-4.77 (m, 2H) 6.94 (br s, 1H) 7.59 (br s, 1H) 10.07 (br s, 1H) 11.28 (br s, 1H). LC/MS: MS (ESI) m/z: 503.1 [M+H+].

The following compounds as shown in Table 2 were prepared using procedures analogous to those described in Example 2 and shown in Scheme 2 using the appropriate starting materials.

TABLE 2

| Compd. | | | Exact Mass |
|---|---|---|---|
| No. | Structure | Name | [M + H]⁺ |
| 6 | 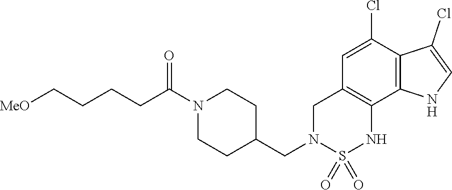 [SMILES: COCCCCC(=O)N1CCC(CN2Cc3cc(Cl)c4c(Cl)c[nH]c4c3NS2(=O)=O)CC1] | 1-(4-((6,7-dichloro- 2,2-dioxido-4,9- dihydro- [1,2,6]thiadiazino[4,3- g]indol-3(1H)- yl)methyl)piperidin-1- yl)-5-methoxypentan- 1-one | Calc'd 503.12, found 503.1 |
| 7 | 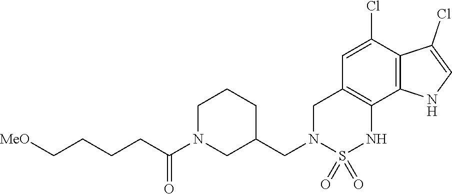 [SMILES: COCCCCC(=O)N1CCCC(CN2Cc3cc(Cl)c4c(Cl)c[nH]c4c3NS2(=O)=O)C1] | 1-(3-((6,7-dichloro- 2,2-dioxido-4,9- dihydro- [1,2,6]thiadiazino[4,3- g]indol-3(1H)- yl)methyl)piperidin-1- yl)-5-methoxypentan- 1-one | Calc'd 503.12, found 503.1 |
| 8 | 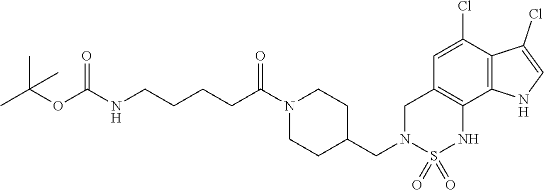 [SMILES: CC(C)(C)OC(=O)NCCCCC(=O)N1CCC(CN2Cc3cc(Cl)c4c(Cl)c[nH]c4c3NS2(=O)=O)CC1] | tert-butyl (5-(4-((6,7- dichloro-2,2-dioxido- 4,9-dihydro- [1,2,6]thiadiazino[4,3- g]indol-3(1H)- yl)methyl)piperidin-1- yl)-5- oxopentyl)carbamate | Calc'd 587.17, found 488.1 (M − Boc + H) |
| 9 | 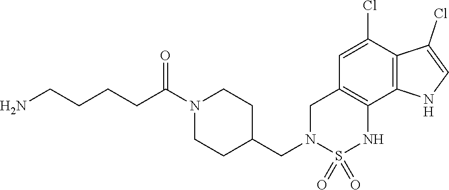 [SMILES: NCCCCC(=O)N1CCC(CN2Cc3cc(Cl)c4c(Cl)c[nH]c4c3NS2(=O)=O)CC1] | 5-amino-1-(4-((6,7- dichloro-2,2-dioxido- 4,9-dihydro- [1,2,6]thiadiazino[4,3- g]indol-3(1H)- yl)methyl)piperidin-1- yl)pentan-1-one | Calc'd 488.12, found 488.1 |

### Example 3

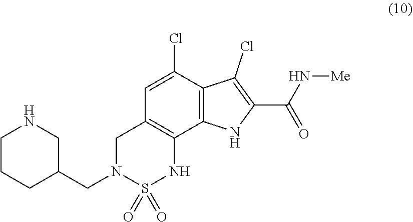 [SMILES: CNC(=O)c1[nH]c2c3c(cc(Cl)c2c1Cl)CN(CC1CCCNC1)S(=O)(=O)N3]

### 6,7-dichloro-N-methyl-3-(piperidin-3-ylmethyl)-1,3,4,9-tetrahydro-[1,2,6]thiadiazino[4,3-g]indole-8-carboxamide 2,2-dioxide (10)

Compound 10 was prepared in an analogous manner to that outlined in Scheme 3.

### Step 1. 6-chloro-8-nitro-1-((2-(trimethylsilyl)ethoxy)methyl)-3,4-dihydro-1H-benzo[c][1,2,6]thiadiazine 2,2-dioxide

To a stirred solution of 6-chloro-8-nitro-3,4-dihydro-1H-benzo[c][1,2,6]thiadiazine 2,2-dioxide (10 g, 37.9 mmol) in DMF (20 ml) was added potassium tert-butoxide (6.38 g, 56.9 mmol) followed by SEM-C1 (8.07 ml, 45.5 mmol) dropwise at 0° C. The mixture was stirred at 20° C. for 17 h. LCMS showed starting material consumed. The mixture was diluted with EtOAc (500 mL), washed with water (150 mL×2) and brine (50 mL), dried over Na2SO4, filtered and concentrated in vacuo to afford the crude, which was purified by flash chromatography (Biotage, C18 column, eluted with MeCN/water (0.1% TFA) from 0 to 75%) to give the compound 6-chloro-8-nitro-1-((2-(trimethylsilyl)ethoxy)methyl)-3,4-dihydro-1H-benzo[c][1,2,6]thiadiazine 2,2-dioxide.

### Step 2, tert-butyl 3-((6-chloro-8-nitro-2,2-dioxido-1-((2-(trimethylsilyl)ethoxy)methyl)-1,4-dihydro-3H-benzo[c][1,2,6]thiadiazin-3-yl)methyl) piperidine-1-carboxylate

To a stirred solution of 6-chloro-8-nitro-1-((2-(trimethylsilyl)ethoxy)methyl)-3,4-dihydro-1H-benzo[c][1,2,6]thiadiazine 2,2-dioxide (2.0 g, 5.08 mmol) in THF (20 ml) was added Ph₃P (6.66 g, 25.4 mmol) and tert-butyl 3-(hydroxymethyl) piperidine-1-carboxylate (1.640 g, 7.62 mmol). The mixture was heated to 70° C. followed by the slow addition of the solution of di-tert-butyl azodicarboxylate (2.92 g, 12.69 mmol) in THF (5 ml) under N₂. The mixture was stirred at 70° C. for 17 h. TLC showed starting material consumed. After cooling down to room temperature, the mixture was concentrated under reduced pressure. The crude product was purified via silica column chromatography (SiO₂, 20% EtOAc in petroleum ether) to afford desired product tert-butyl 3-((6-chloro-8-nitro-2,2-dioxido-1-((2-(trimethylsilyl)ethoxy)methyl)-1,4-dihydro-3H-benzo[c][1,2,6]thiadiazin-3-yl)methyl) piperidine-1-carboxylate.

Step 3, tert-butyl 3-((6-chloro-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate

To a stirred solution of tert-butyl 3-((6-chloro-8-nitro-2,2-dioxido-1-((2-(trimethylsilyl)ethoxy)methyl)-1,4-dihydro-3H-benzo[c][1,2,6]thiadiazin-3-yl)methyl) piperidine-1-carboxylate (2 g, 3.38 mmol) in THF (20 ml) was added vinylmagnesium bromide (16.91 ml, 16.91 mmol) [1.0M in THF]dropwise at −78° C. The mixture was then stirred at −40° C. for 2 h. LCMS showed starting material consumed and desired product formed. The mixture was quenched with saturated NH₄Cl solution (150 ml) then extracted with EtOAc (150 mL×2). The organic phase was concentrated under reduced pressure. The crude product was purified via CombiFlash® (Biotage, 20 g silica gel, eluted with EtO Ac/petroleum ether from 0 to 10%) to afford desired product tert-butyl 3-((6-chloro-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate.

### Step 4, tert-butyl 3-((6,7-dichloro-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate

The solution of tert-butyl 3-((6-chloro-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate (1-3, 500 mg, 0.854 mmol) and NCS (125 mg, 0.940 mmol) in THF (5 ml) and DMF (0.2 ml) was stirred at 25° C. for 17 h. LCMS showed formation of product. The mixture was purified by CombiFlash® (Biotage, 12 g silica gel, eluted with EtOAc/petroleum ether from 0 to 10%) to give the desired product tert-butyl 3-((6,7-dichloro-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate.

### Step 5, tert-butyl 3-((6,7-dichloro-8-iodo-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate

The solution of tert-butyl 3-((6,7-dichloro-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate (300 mg, 0.484 mmol) and NIS (218 mg, 0.968 mmol) in THF (3 ml) and DMF (0.1 ml) was stirred at 25° C. for 17 h. The mixture was purified by CombiFlash® (Biotage, 12 g silica gel, eluted with EtO Ac/petroleum ether from 0 to 10%) to give the desired product tert-butyl 3-((6,7-dichloro-8-iodo-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate.

### Step 6, tert-butyl 3-((6,7-dichloro-8-(methylcarbamoyl)-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate

To the solution of tert-butyl 3-((6,7-dichloro-8-iodo-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate (200 mg, 0.268 mmol) and TEA (0.037 ml, 0.268 mmol) in DMF (3 ml) was added methanamine (278 mg, 2.68 mmol) [30% in EtOH] followed by PdCl₂ (dppf)-CH₂Cl₂ adduct (21.91 mg, 0.027 mmol) under Ar. Then the mixture was stirred at 70° C. for 17 h under CO (40 psi). The mixture was purified by prep-TLC (ether/EtOAc=4/1) to give the desired product tert-butyl 3-((6,7-dichloro-8-(methylcarbamoyl)-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate.

### Step 7, tert-butyl 3-((6,7-dichloro-8-(methylcarbamoyl)-2,2-dioxido-4,9-dihydro-[1,2,6]thiadiazino[4,3-g]indol-3 (1H)-yl)methyl) piperidine-1-carboxylate

To a stirred solution of tert-butyl 3-((6,7-dichloro-8-(methylcarbamoyl)-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate (100 mg, 0.148 mmol) in THF (2 ml) was added TBAF (1.478 ml, 1.478 mmol) [1.0 Min THF]. The mixture was then stirred at 70° C. for 17 h. The mixture was purified by prep-HPLC (TFA) to give desired product tert-butyl 3-((6,7-dichloro-8-(methylcarbamoyl)-2,2-dioxido-4,9-dihydro-[1,2,6]thiadiazino[4,3-g]indol-3 (1H)-yl)methyl) piperidine-1-carboxylate.

### Step 8. 6,7-dichloro-N-methyl-3-(piperidin-3-ylmethyl)-1,3,4,9-tetrahydro-[1,2,6]thiadiazino[4,3-g]indole-8-carboxamide 2,2-dioxide

To a stirred solution of tert-butyl 3-((6,7-dichloro-8-(methylcarbamoyl)-2,2-dioxido-4,9-dihydro-[1,2,6]thiadiazino[4,3-g]indol-3 (1H)-yl)methyl) piperidine-1-carboxylate (1-7, 50 mg, 0.091 mmol) in DCM (1.5 ml) was added TFA (0.5 ml, 0.091 mmol). The mixture was stirred at 25° C. for 2 h. The mixture was concentrated under reduced pressure, then suspended in methyl t-butyl ether (1 mL) and filtered to afford desired product Compound 10. LCMS (ESI) m/z: 446.0 [M+H]⁺.

The following compounds as shown in Table 3 were prepared using procedures analogous to those described for Compound 10 (Scheme 3) using the appropriate starting materials.

TABLE 3

| | | | Exact Mass |
|---|---|---|---|
| Compd. | | | [M + H]⁺ or |
| No. | Structure | Name | [M − H]⁻ |
| 11 | 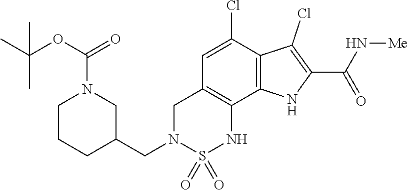 [SMILES: CNC(=O)c1[nH]c2c3c(cc(Cl)c2c1Cl)CN(CC1CCCN(C(=O)OC(C)(C)C)C1)S(=O)(=O)N3] | tert-butyl 3-((6,7- dichloro-8- (methylcarbamoyl)- 2,2-dioxido-4,9- dihydro- [1,2,6]thiadiazino[4,3- g]indol-3(1H)- yl)methyl)piperidine- 1-carboxylate | Calc'd 546.13, found 546.1 |
| 12 | 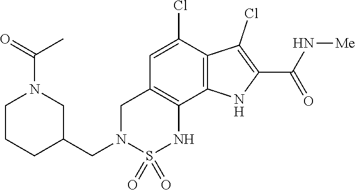 [SMILES: CNC(=O)c1[nH]c2c3c(cc(Cl)c2c1Cl)CN(CC1CCCN(C(C)=O)C1)S(=O)(=O)N3] | 3-((1-acetylpiperidin- 3-yl)methyl)-6,7- dichloro-N-methyl- 1,3,4,9-tetrahydro- [1,2,6]thiadiazino[4,3- g]indole-8- carboxamide 2,2- dioxide | Calc'd 488.08, found 488.1 |
| 13 | 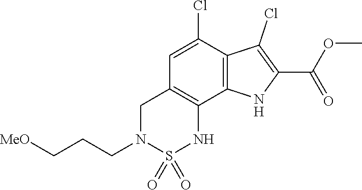 [SMILES: COCCCN1Cc2cc(Cl)c3c(Cl)c(C(=O)OC)[nH]c3c2NS1(=O)=O] | methyl 6,7-dichloro- 3-(3-methoxypropyl)- 1,3,4,9-tetrahydro- [1,2,6]thiadiazino[4,3- g]indole-8- carboxylate 2,2- dioxide | Calc'd 422.03, found 422.1 |
| 14 | 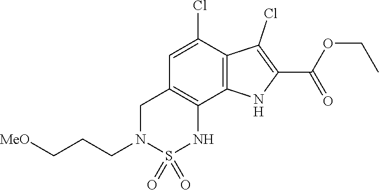 [SMILES: CCOC(=O)c1[nH]c2c3c(cc(Cl)c2c1Cl)CN(CCCOC)S(=O)(=O)N3] | ethyl 6,7-dichloro-3- (3-methoxypropyl)- 1,3,4,9-tetrahydro- [1,2,6]thiadiazino[4,3- g]indole-8- carboxylate 2,2- dioxide | Calc'd 436.04, found 436.0 |
| 15 | 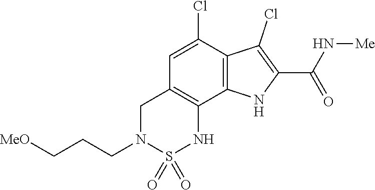 [SMILES: CNC(=O)c1[nH]c2c3c(cc(Cl)c2c1Cl)CN(CCCOC)S(=O)(=O)N3] | 6,7-dichloro-3-(3- methoxypropyl)-N- methyl-1,3,4,9- tetrahydro- [1,2,6]thiadiazino[4,3- g]indole-8- carboxamide 2,2- dioxide | Calc'd 421.04, found 421.0 |
| 16 | 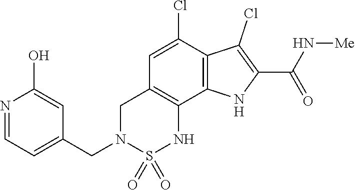 [SMILES: CNC(=O)c1[nH]c2c3c(cc(Cl)c2c1Cl)CN(Cc1ccnc(O)c1)S(=O)(=O)N3] | 6,7-dichloro-3-((2- hydroxypyridin-4- yl)methyl)-N-methyl- 1,3,4,9-tetrahydro- [1,2,6]thiadiazino[4,3- g]indole-8- carboxamide 2,2- dioxide | Calc'd 456.02, found 456.0 |

### Example 4

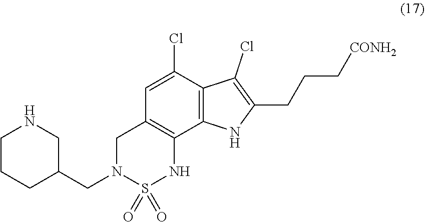 [SMILES: NC(=O)CCCc1[nH]c2c3c(cc(Cl)c2c1Cl)CN(CC1CCCNC1)S(=O)(=O)N3]

### 4-(6,7-dichloro-2,2-dioxido-3-(piperidin-3-ylmethyl)-1,3,4,9-tetrahydro-[1,2,6]thiadiazino[4,3-g]indol-8-yl)butanamide (17)

Compound 17 was prepared in an analogous manner to that outlined in Scheme 4.

### Step 1, tert-butyl 3-((6-chloro-8-(4-methoxy-4-oxobutyl)-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate

To a stirred solution of tert-butyl 3-((6-chloro-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate (100 mg, 0.171 mmol) and methyl 4-bromobutanoate (93 mg 0.513 mmol) in DMA (2 ml) and water (0.02 ml) was added K₂CO₃ (47.2 mg, 0.342 mmol), bicyclo[2.2.1]hept-2-ene (32.2 mg, 0.342 mmol) and palladium (II) chloride (6.06 mg, 0.034 mmol) under N₂. The mixture was stirred at 70° C. for 17 h. The mixture was purified by prep-HPLC (TFA) to afford desired product tert-butyl 3-((6-chloro-8-(4-methoxy-4-oxobutyl)-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate.

### Step 2, tert-butyl 3-((6,7-dichloro-8-(4-methoxy-4-oxobutyl)-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate

The solution of tert-butyl 3-((6-chloro-8-(4-methoxy-4-oxobutyl)-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate (30 mg, 0.044 mmol) and NCS (7.01 mg, 0.053 mmol) in THF (1 ml) and DMF (0.02 ml) was stirred at 25° C. for 17 h. The mixture was diluted with EtOAc (10 mL), washed with aq. NaS₂O₃ (2 mL) and brine (2 mL), dried over Na₂SO₄. The mixture was filtered and concentrated to give the crude compound tert-butyl 3-((6,7-dichloro-8-(4-methoxy-4-oxobutyl)-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate, which was used in next step without further purification.

### Step 3. 4-(3-((1-(tert-butoxycarbonyl) piperidin-3-yl)methyl)-6,7-dichloro-2,2-dioxido-1-((2-(trimethylsilyl)ethoxy)methyl)-1,3,4,9-tetrahydro-[1,2,6]thiadiazino[4,3-g]indol-8-yl)butanoic acid

The solution of tert-butyl 3-((6,7-dichloro-8-(4-methoxy-4-oxobutyl)-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate (25 mg, 0.035 mmol) and LiOH (0.069 ml, 0.069 mmol) [1.0 M in water] in THF (0.5 ml) and MeOH (0.5 ml) was stirred at 25° C. for 5 h. The mixture was concentrated and acidified to pH-3 with aq. 1 N HCl solution, extracted with EtOAc (10 mL), then the organic phase was washed with brine (5 mL), dried over Na₂SO₄, filtered and concentrated to give the crude compound 4-(3-((1-(tert-butoxycarbonyl) piperidin-3-yl)methyl)-6,7-dichloro-2,2-dioxido-1-((2-(trimethylsilyl)ethoxy)methyl)-1,3,4,9-tetrahydro-20) [1,2,6]thiadiazino[4,3-g]indol-8-yl)butanoic acid, which was used in next step without further purification.

### Step 4, tert-butyl 3-((8-(4-amino-4-oxobutyl)-6,7-dichloro-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate

To the solution of 4-(3-((1-(tert-butoxycarbonyl) piperidin-3-yl)methyl)-6,7-dichloro-2,2-dioxido-1-((2-(trimethylsilyl)ethoxy)methyl)-1,3,4,9-tetrahydro-[1,2,6]thiadiazino[4,3-g]indol-8-yl)butanoic acid (30 mg, 0.030 mmol) and ammonium chloride (4.77 mg, 0.089 mmol) in DMF (1 ml) was added DIEA (0.026 ml, 0.149 mmol) followed by HATU (16.97 mg, 0.045 mmol), then the mixture was stirred at 25° C. for 17 h. The mixture was purified by prep-TLC (DCM/MeOH=15/1) to give the desired product tert-butyl 3-((8-(4-amino-4-oxobutyl)-6,7-dichloro-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate.

### Step 5, tert-butyl 3-((8-(4-amino-4-oxobutyl)-6,7-dichloro-2,2-dioxido-4,9-dihydro-[1,2,6]thiadiazino[4,3-g]indol-3 (1H)-yl)methyl) piperidine-1-carboxylate

To the solution of tert-butyl 3-((8-(4-amino-4-oxobutyl)-6,7-dichloro-2,2-dioxido-1-((2-(trimethylsilyl)ethoxy)methyl)-4,9-dihydro-[1,2,6]thiadiazino[4,3-g]indol-3 (1H)-yl)methyl) piperidine-1-carboxylate (50 mg, 0.071 mmol) and THF (1 ml) was added TBAF (0.709 ml, 0.709 mmol), the mixture was stirred at 70° C. for 17 h. The mixture was purified by TLC (DCM/MeOH=10/1) to give the desired product tert-butyl 3-((8-(4-amino-4-oxobutyl)-6,7-dichloro-2,2-dioxido-4,9-dihydro-[1,2,6]thiadiazino[4,3-g]indol-3 (1H)-yl)methyl) piperidine-1-carboxylate.

### Step 6. 4-(6,7-dichloro-2,2-dioxido-3-(piperidin-3-ylmethyl)-1,3,4,9-tetrahydro-[1,2,6]thiadiazino[4,3-g]indol-8-yl)butanamide

The solution of tert-butyl 3-((8-(4-amino-4-oxobutyl)-6,7-dichloro-2,2-dioxido-4,9-dihydro-[1,2,6]thiadiazino[4,3-g]indol-3 (1H)-yl)methyl) piperidine-1-carboxylate (2-5, 5 mg, 8.70 μmol) in DCM (0.5 ml) and TFA (0.1 ml) was stirred at 20° C. for 17 h. The mixture was concentrated and purified by prep-HPLC (TFA) to afford desired product compound 17. ¹H NMR (400 MHZ, METHANOL-d4) δ 6.84 (s, 1H), 4.72 (s, 2H), 3.52 (brd, J=13.45 Hz, 1H), 3.39 (br s, 1H), 2.89-2.98 (m, 3H), 2.85 (t, J=7.46 Hz, 2H), 2.75 (t, J=12.10 Hz, 1H), 2.23-2.31 (m, 2H), 2.16 (br d, J=5.62 Hz, 1H), 1.96-2.05 (m, 3H), 1.91 (br d, J=12.96 Hz, 1H), 1.68-1.83 (m, 1H), 1.26-1.35 (m, 1H). LCMS (ESI) m/z: 474.0 [M+H]⁺.

The following compounds 18-26, depicted in Table 4, were prepared using procedures similar to those described for compound 17 (Scheme 4) using the appropriate starting materials.

TABLE 4

| | | | Exact Mass |
|---|---|---|---|
| Compds. | | | [M + H]⁺ or |
| No. | Structure | Name | [M − H]⁻ |
| 18 | 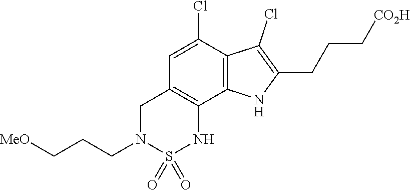 [SMILES: COCCCN1Cc2cc(Cl)c3c(Cl)c(CCCC(=O)O)[nH]c3c2NS1(=O)=O] | 4-(6,7-dichloro-3-(3- methoxypropyl)-2,2- dioxido-1,3,4,9- tetrahydro- [1,2,6]thiadiazino[4,3- g]indol-8-yl)butanoic acid | Calc'd 450.06, found 450.0 |
| 19 | 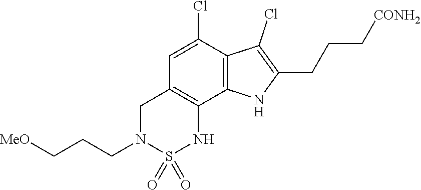 [SMILES: COCCCN1Cc2cc(Cl)c3c(Cl)c(CCCC(N)=O)[nH]c3c2NS1(=O)=O] | 4-(6,7-dichloro-3-(3- methoxypropyl)-2,2- dioxido-1,3,4,9- tetrahydro- [1,2,6]thiadiazino[4,3- g]indol-8- yl)butanamide | Calc'd 449.07, found 449.0 |
| 20 | 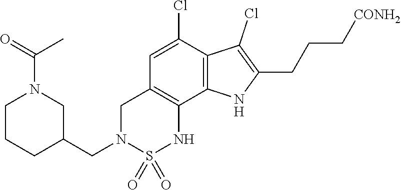 [SMILES: CC(=O)N1CCCC(CN2Cc3cc(Cl)c4c(Cl)c(CCCC(N)=O)[nH]c4c3NS2(=O)=O)C1] | 4-(3-((1- acetylpiperidin-3- yl)methyl)-6,7- dichloro-2,2-dioxido- 1,3,4,9-tetrahydro- [1,2,6]thiadiazino[4,3- g]indol-8- yl)butanamide | Calc'd 516.12, found 516.1 |
| 21 | 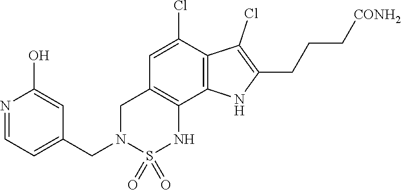 [SMILES: NC(=O)CCCc1[nH]c2c3c(cc(Cl)c2c1Cl)CN(Cc1ccnc(O)c1)S(=O)(=O)N3] | 4-(6,7-dichloro-3-((2- hydroxypyridin-4- yl)methyl)-2,2- dioxido-1,3,4,9- tetrahydro- [1,2,6]thiadiazino[4,3- g]indol-8- yl)butanamide | Calc'd 484.05, found 484.0 |
| 22 | 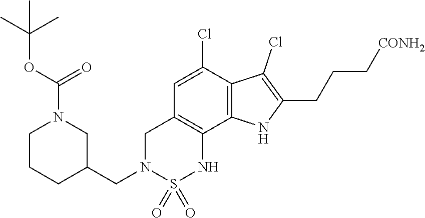 [SMILES: CC(C)(C)OC(=O)N1CCCC(CN2Cc3cc(Cl)c4c(Cl)c(CCCC(N)=O)[nH]c4c3NS2(=O)=O)C1] | tert-butyl 3-((8-(4- amino-4-oxobutyl)- 6,7-dichloro-2,2- dioxido-4,9-dihydro- [1,2,6]thiadiazino[4,3- g]indol-3(1H)- yl)methyl)piperidine- 1-carboxylate | Calc'd 573.16, found 474.1 [M − Boc + H]+. |
| 23 | 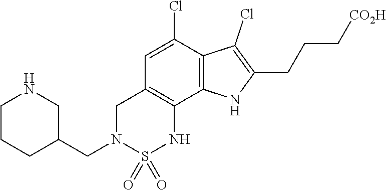 [SMILES: O=C(O)CCCc1[nH]c2c3c(cc(Cl)c2c1Cl)CN(CC1CCCNC1)S(=O)(=O)N3] | 4-(6,7-dichloro-2,2- dioxido-3-(piperidin- 3-ylmethyl)-1,3,4,9- tetrahydro- [1,2,6]thiadiazino[4,3- g]indol-8-yl)butanoic acid | Calc'd 474.09, found 475.0 |
| 24 | 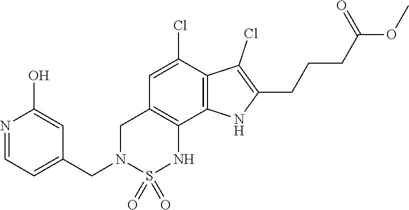 [SMILES: COC(=O)CCCc1[nH]c2c3c(cc(Cl)c2c1Cl)CN(Cc1ccnc(O)c1)S(=O)(=O)N3] | methyl 4-(6,7- dichloro-3-((2- hydroxypyridin-4- yl)methyl)-2,2- dioxido-1,3,4,9- tetrahydro- [1,2,6]thiadiazino[4,3- g]indol-8- yl)butanoate | Calc'd 498.05, found 499.0 |
| 25 | 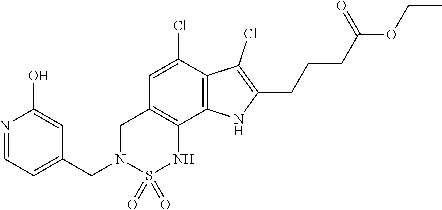 [SMILES: CCOC(=O)CCCc1[nH]c2c3c(cc(Cl)c2c1Cl)CN(Cc1ccnc(O)c1)S(=O)(=O)N3] | ethyl 4-(6,7-dichloro- 3-((2-hydroxypyridin- 4-yl)methyl)-2,2- dioxido-1,3,4,9- tetrahydro- [1,2,6]thiadiazino[4,3- g]indol-8- yl)butanoate | Calc'd 512.07, found 513.1 |
| 26 | 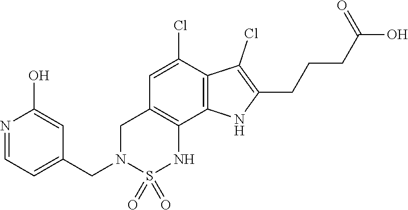 [SMILES: O=C(O)CCCc1[nH]c2c3c(cc(Cl)c2c1Cl)CN(Cc1ccnc(O)c1)S(=O)(=O)N3] | 4-(6,7-dichloro-3-((2- hydroxypyridin-4- yl)methyl)-2,2- dioxido-1,3,4,9- tetrahydro- [1,2,6]thiadiazino[4,3- g]indol-8-yl)butanoic acid | Calc'd 484.04, found 485.1 |

### Assays

The compounds of the present invention were subjected to various assays to determine their biological activity. A description of the assays which may be used are presented below. Additionally, selected assay results for particular compounds of the present invention are presented.

Proteins used for the assays were as follows: Human DDA1 (UniProt:Q9BW61), DDB1 (UniPro:Q16531), and DCAF15 (UniProt:Q66K64) coding sequences were cloned into pFastBac1 vectors and were co-expressed in Sf9 cells using Bac-to-Bac baculovirus expression system (Thermo Fisher Scientific). The expression construct for DCAF15 includes a N-terminal His6-tag to facilitate the purification. The atrophin-1 homology region of DCAF15 (amino acid residues 276-383) was excised (Apro, originally to facilitate crystallization). Triplex of DDA1-DDB1ΔB-DCAF15Apro, which has the BPB domain (aa 396 to 705) excised and replaced with a GNGNSG linker, was used in all the kinetic measurements because of the higher yield. R¹R² of RBM39 (aa 150 to 331; UniProt: Q14498) contained a C-terminal FLAG tag used in the DCAF15/RBM39 complex formation assay. DCAF15 Displacement Assay (Displacement FRET K_{D})

The previously described procedure was followed (See X. Du, et al., “Structural Basis and Kinetic Pathway of RBM39 Recruitment to DCAF15 by a Sulfonamide Molecular Glue E7820”, Structure, 27, 1625-1633.e3, 2019). Synthesis of the probe PT7795 is described in the article.

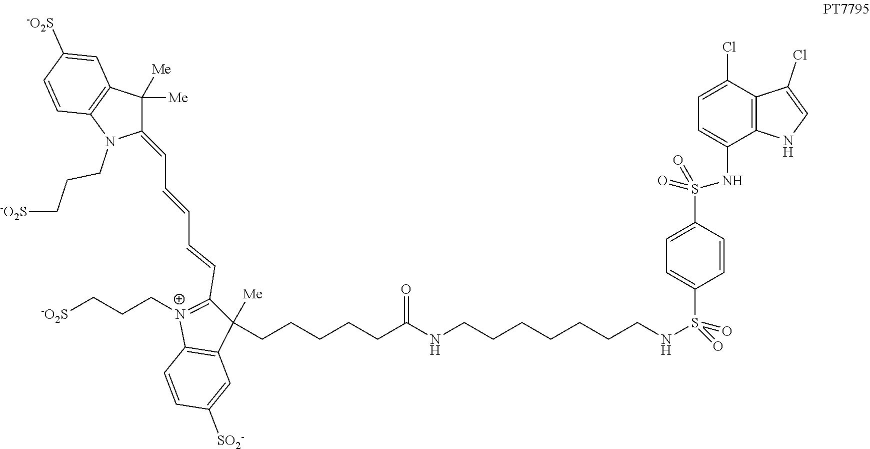

Binding of PT7795 to DDA1-DDB1ΔB-DCAF15Apro was measured by the FRET signal between europium-labeled anti-His antibody (AD0205, Perkin Elmer) that is associated with His-tagged DCAF15 and ALX647 moiety in PT7795. The concentration of PT7795 was varied from 2.3 nM to 1000 nM in 16 wells, each containing 25 mM HEPES pH7.5, 100 mM NaCl, 0.1 mg/ml BSA, 0.005% Tween 20, 0.5 mM TCEP, 4% DMSO, 0.5 nM europium-labeled anti-His antibody, and 10 nM triplex of DDA1-DDB1 DB-DCAF15 Apro. After equilibrating for 2-3 hours at 4° C., the plate was removed from the incubator, briefly spun, then read on a Spark 10 (Tecan) at the excitation/emission wavelength of 340 nm/615 nm and 340 nm/665 nm. To obtain K_{d}, the ratio of 665/615 readings was fitted to Equation 1, where F stands for the ratio of 665/615 nM.

$$F = {F}_{max} [ PT 7795 ] / ( {K}_{d} + [ PT 7795 ] ) \qquad ( Equation 1 )$$

Binding of compounds in this disclosure to DCAF15 was measured in a competition by the displacement of PT7795. The concentration of PT7795 was kept at 300 nM while the concentration of a compound was varied from 0.01 to 100 μM. The incubation time was increased to 18 hours for enhanced signal to noise level. To obtain IC₅₀, the ratio of 665/615 readings was fitted to Equation 2, where n is Hill slope.

$$F = {F}_{min} + ( {F}_{max} - {F}_{min} ) / ( 1 + {10}^{( logIC 50 - log [ Compound ] ) n} ) \qquad ( Equation 2 )$$

The K_{d}s can then be calculated from IC₅₀s using Equation 3 (Cheng-Prusoff equation).

$${K}_{d} = {IC}_{50} / ( 1 + ( [ PT 7795 ] / {{K}_{d}}^{PT 7795} ) ) \qquad ( Equation 3 )$$

### DCAF15 RBM39 Ternary Complex Formation Assay

His-tagged DDA1-DDB1ΔB-DCAFISΔpro triplex and RIR2 with a C-terminal FLAG were used in the assay and compound-induced complex formation was monitored through resonance transfer between europium-labeled anti-His antibody and APC-labeled anti-FLAG antibody (AD0059F, Perkin Elmer). Assay buffer contained 25 mM HEPES, pH7.5, 100 mM NaCl, 0.1 mg/ml BSA, 0.005% Tween 20, 0.5 mM TCEP, 0.5 nM europium-labeled anti-His antibody, 50 nM APC-labeled anti-FLAG antibody, 10 nM triplex of DCAF15, and 50 nM RIR2 The concentration of the tested compound was varied and the ratio of 665/615 nm readings was fitted to Equation 2 to obtain the apparent EC₅₀ for compound-induced complex formation.

### CellTiter-Glo Assay

HCT116 human colorectal carcinoma cells were used in these experiments. HCT116 cells were obtained from ATCC. CellTiter-Glo® Luminescent Cell Viability Assay Reagent was obtained from Promega Corporation, Madison, Wisconsin, USA.

About 3,000 cells were seeded into 96-well plate with 100 μL of media (DMEM supplemented with 10% FBS, 100 units penicillin, and 100 mg streptomycin per mL) the day before the experiment. For compound treatment, the master plate was prepared as follows: 100× compound stocks were prepared in a 96-well plate by 1 to 3 serial dilutions of 0.5 mM DMSO stocks. Each master plate contained serial dilution of 8 compounds, including the control compound E7820. 1 mL of 100× compound stock was added to each well of the assay plate to give final concentrations of 5, 1.67, 0.56, 0.19, 0.06, 0.02, 0.0069, 0.002, 0.0007, 0.0002, and 0 mM. Each concentration was tested in triplicate. After 3 days, cell viability was determined using CellTiter-Glo Luminescent Cell Viability Assay Reagent following the manufacturer's recommended protocol. Briefly, 50 mL of CellTiter-Glo reagent was added to each well of the assay plate and the contents were mixed for 3 minutes at 600 rpm on a shaker (Thermomixer R). The plate was immediately placed in a plate reader (Synergy 2) and the luminescence signal (0.5 second per well integration time) was determined. IC₅₀ values were calculated using Dotmatics software (Dotmatics, Bishops Stortford, UK)

Table 5 depicts biological activities, Displacement IC₅₀ and DCAF15 RBM39 Ternary Complex Formation EC₅₀, of selected compounds described herein in the complex formation. Compound numbers correspond to the numbers and structures provided in Table 1-4 and Examples.

TABLE 5

| | Displace- | Ternary | | Displace- | Ternary |
|---|---|---|---|---|---|
| Compound | ment IC₅₀ | complex | Compound | ment IC₅₀ | complex |
| No. | (nM) | EC₅₀ (nM) | No. | (nM) | EC₅₀ (nM) |
| 1 | 1419 | 12.7 | 14 | 2054 | 2509 |
| 2 | 25000 | 247 | 15 | 1280 | >25000 |
| 3 | 2497 | 21.4 | 18 | 652 | >25000 |
| 4 | 200 | 4.3 | 19 | 824 | >25000 |
| 5 | 98 | 7.8 | 16 | NA | NA |
| 6 | 156 | 4.3 | 17 | NA | NA |
| 7 | 480 | 10.6 | 20 | 105 | >25000 |
| 8 | 261 | 4.5 | 21 | NA | NA |
| 9 | 244 | 4.9 | 22 | 5778 | >25000 |
| 10 | 2088 | >25000 | 23 | 277 | >25000 |
| 11 | 1444 | >25000 | 24 | 317 | >25000 |
| 12 | 679 | >25000 | 25 | NA | NA |
| 13 | 1296 | >25000 | 26 | NA | NA |

### DCAF15 RBM39 Complex Formation Assay

LANCE Eu-W1024 Anti-6×His and SureLight Allophycocyanin-anti-FLAG antibodies were purchased from Perkin Elmer. Human DDB1 DB and His-tagged DCAF15 were co-expressed in baculovirus/sf9 system. While full length DDB1 contains three 7-bladed propellers termed A, B, and C, DDB1 DB has only propeller A (amino acids 1 to 395) and C (amino acids 709-1140). The His-tagged DCAF15 contains six histidine residues directly preceding the DCAF15 sequence without any linker. An RBM39 fragment containing RRM1 and RRM2 was expressed in E. coli. Amino acids 150 to 331 of RBM39 were cloned into pGEX4T3 with an N-terminal TEV cleavage site and a C-terminal FLAG tag.

A compound of the present disclosure in DMSO (2 μL) was transferred to a 384-well assay plate. Compound was added in 14 points, 3-fold serial dilution (1 μM to 0.6 pico molar) in DMSO. 48 mL FRET assay buffer (25 mM HEPES, pH 7.5, 100 mM NaCl, 0.1 MBSA, 0.5 mM TCEP, 0.005% Tween-20, 0.2 nM Eu—W1024 Anti-6×His, 50 nM SureLight Allophycocyanin-anti-FLAG, 20 nM DDB1 DB/DCAF15, 50 nM RBM39 R¹R²) was added and the resulting solution was incubated overnight at 4° C. The plate was placed in a plate reader (Spark 10) with excitation at 1=340 nm and emissions monitored at 1=665 nm and 1=615 nm, and the FRET ratio (Em 665/Em 615) was determined. Low signal controls containing no compound and high signal controls containing 10 mM indisulam were measured for each plate.

EC₅₀ values were calculated using Dotmatics template equation for 4-parameter fit. Results are expressed as % formation using the following equation:

$$\% formation = ( \frac{high control - sample}{high control - low control} ) \times 100$$

1. A compound of Formula I 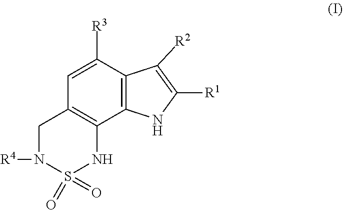
or a pharmaceutically acceptable salt thereof, wherein:
R¹ is selected from H, (C₁-C₆)alkyl, aminocarbonyl(C₁-C₆)alkyl, (C₁-C₆)alkylaminocarbonyl(C₀-C₆)alkyl, carboxy (C₁-C₆)alkyl, hydroxycarbonyl(C₀-C₆)alkyl), and (C₁-C₆)alkoxycarbonyl(C₀-C₆)alkyl);
R² is H, (C₁-C₆)alkyl, cyano, —(C₁-C₆) OH, halogen, or (C₁-C₆)haloalkyl;
R³ is H, (C₁-C₆)alkyl, cyano, —(C₁-C₆) OH, halogen, or (C₁-C₆)haloalkyl;
R⁴ is selected from (C₁-C₆)alkoxy ((C₁-C₆)alkyl), heterocycloalkyl((C₀-C₆)alkyl), heteroaryl((C₀-C₆)alkyl), —C(═O) (C₁-C₆)alkyl-(O—(C₁-C₆)alkyl)ₙ, and —(C₁-C₆)alkyl-(O—(C₁-C₆))ₙ, wherein R⁴ is substituted with 0, 1, 2, or 3 R⁵ substituents;
each n is independently 1, 2, 3, or 4;
each R⁵ independently is selected from cyano, (C₁-C₆)haloalkyl, halogen, hydroxy, oxo, (C₁-C₆)alkyl, (C₁-C₆)alkoxy ((C₀-C₆)alkyl), —SO₂ (C₁-C₆)alkyl, amino, —C(═O) (C₁-C₆)alkyl-(O—(C₁-C₆)alkyl)ₘ, —(C₁-C₆)alkyl-(O—(C₁-C₆)alkyl)ₘ, amino (C₁-C₆)akylcarbonyl, (C₁-C₆)alkylcarboxy, (C₁-C₆)alkylcarbonyl, and —(C₁-C₆)alkylOH, wherein each R⁵ independently is substituted with 0, 1, 2, or 3 R⁶ substituents;
each R⁶ is independently selected from amino, (C₁-C₆)alkylcarboxy, (C₁-C₄)alkyl, halogen and hydroxy;
each m independently is 1, 2, or 3; and provided that when R⁴ is (C₁-C₆)alkoxy ((C₁-C₆)alkyl), heterocycloalkyl((C₀-C₆)alkyl), or heteroaryl((C₀-C₆)alkyl) then R¹ is other than hydrogen.

2. A compound of claim 1 or a pharmaceutically acceptable salt thereof, wherein R¹ is selected from hydrogen, aminocarbonyl(C₁-C₆)alkyl, (C₁-C₆)alkylaminocarbonyl(C₀-C₆)alkyl, carboxy (C₁-C₆)alkyl, hydroxycarbonyl(C₀-C₆)alkyl), and (C₁-C₆)alkoxycarbonyl(C₀-C₆)alkyl).

3. A compound of claim 2 or a pharmaceutically acceptable salt thereof, wherein R¹ is selected from hydrogen, aminocarbonylmethyl, aminocarbonylethyl, methylaminocarbonyl, ethylaminocarbonyl, propylaminocarbonyl, methylcarboxy, ethylcarboxy, carboxypropyl, carboxyisopropyl carboxy butyl, aminocarbonylpropyl, aminocarbonylethyl, and aminocarbonylmethyl.

4. A compound of claim 3 or a pharmaceutically acceptable salt thereof, wherein R¹ is selected from hydrogen, methylaminocarbonyl, methylcarboxy, ethylcarboxy, carboxypropyl, and aminocarbonylpropyl.

5. A compound of claim 4 or a pharmaceutically acceptable salt thereof, wherein R² is hydrogen, halogen, or (C₁-C₆)haloalkyl.

6. A compound of claim 5 or a pharmaceutically acceptable salt thereof, wherein R² is hydrogen, chloro, fluoro or bromo.

7. A compound of claim 6 or a pharmaceutically acceptable salt thereof, wherein R³ is hydrogen, halogen, or (C₁-C₆)haloalkyl.

8. A compound of claim 7 or a pharmaceutically acceptable salt thereof, wherein R³ is hydrogen, chloro, fluoro or bromo.

9. A compound of claim 8 or a pharmaceutically acceptable salt thereof, wherein R⁴ is selected from (C₁-C₄)alkoxy ((C₁-C₄)alkyl), heterocycloalkyl((C₀-C₄)alkyl), heteroaryl((C₀-C₄)alkyl), —C(═O) (C₁-C₆)alkyl-(O—(C₁-C₆)alkyl)ₙ, and —(C₁-C₆)alkyl-(O—(C₁-C₆alkyl))ₙ, wherein R⁴ is substituted with 0, 1, 2, or 3 R⁵ substituents.

10. A compound of claim 1 or a pharmaceutically acceptable salt thereof, wherein R⁴ is selected from methoxyethyl, pyridyl, pyridylmethyl, (1,2-dihydropyridyl)methyl, pyrimidylmethyl, oxetanylmethyl, (oxadiazolyl)methyl, (1,3,4-oxadiazolyl)methyl, triazolylmethyl, (1,2,4-triazolyl)methyl, (piperidinyl)methyl, pyridazinylmethyl, pyrazolylmethyl, tetrahydropyranylmethyl, (tetrahydro-2H-pyranyl)methyl, azetidinylmethyl, (1,2-dihydropyridinyl)methyl, pyrazinylmethyl, 3-methoxypropyl, morpholinylethyl, tetrahydro-2H-pyranyl, tetrahydropyranyl, pyridinyl, (tetrahydrofuranyl)methyl, oxetanylethyl, 3-(2-methoxyethoxy)propyl, 3-((2-2-methoxyethoxy)ethoxy)propyl, methoxypropyl, (methoxyethoxy)methylcarbonyl, 4-methoxybutylcarbonyl, and 3-(methoxypentoxy)propyl, wherein R⁴ is substituted with 0, 1, 2, or 3 R⁵ substituents.

11. A compound of claim 10 or a pharmaceutically acceptable salt thereof, wherein each R⁵ independently is selected from (C₁-C₆)alkylcarbonyl, hydroxy, —C(═O) (C₁-C₆)alkyl-(O—(C₁-C₆)alkyl)ₘ, —(C₁-C₆)alkyl-(O—(C₁-C₆)alkyl)ₘ, amino (C₁-C₆)akylcarbonyl, (C₁-C₆)alkylcarbonyl, and (C₁-C₆)alkylcarboxy, wherein each R⁵ independently is substituted with 0, 1, 2, or 2 R⁶ substituents.

12. A compound of claim 11 or a pharmaceutically acceptable salt thereof, wherein each R⁵ independently is selected from (2-methoxyethoxy)methylcarbonyl, tert-butylcarboxy, 4-methoxybutylcarbonyl, methylcarbonyl, hydroxy, 4-aminobutylcarbonyl, wherein each R⁵ independently is substituted with 0, 1, 2, or 3 R⁶.

13. A compound of to claim 12 or a pharmaceutically acceptable salt thereof, wherein each R⁶ is independently selected from amino, tert-butylcarboxy, methylcarboxy, ethylcarboxy, methyl, ethyl, propyl, isopropyl, fluoro, chloro and hydroxy.

14. A compound of claim 13 or a pharmaceutically acceptable salt thereof, wherein each R⁶ is independently amino, or tert-butylcarboxy.

15. A compound of claim 14 or a pharmaceutically acceptable salt thereof, wherein each n is independently 1, 2, or 3.

16. A compound of claim 15 or a pharmaceutically acceptable salt thereof, wherein each m independently is 1, or 2.

17. A compound of claim 1 or a pharmaceutically acceptable salt thereof, selected from:
6,7-dichloro-3-(3-(2-methoxyethoxy)propyl)-1,3,4,9-tetrahydro-[1,2,6]thiadiazino[4,3-g]indole 2,2-dioxide;
6,7-dichloro-3-(3-(2-(2-methoxyethoxy)ethoxy)propyl)-1,3,4,9-tetrahydro-[1,2,6]thiadiazino[4,3-g]indole 2,2-dioxide;
6,7-dichloro-3-(3-((5-methoxypentyl)oxy)propyl)-1,3,4,9-tetrahydro-[1,2,6]thiadiazino[4,3-g]indole 2,2-dioxide;
1-(4-((6,7-dichloro-2,2-dioxido-4,9-dihydro-[1,2,6]thiadiazino[4,3-g]indol-3 (1H)-yl)methyl) piperidin-1-yl)-2-(2-methoxyethoxy)ethan-1-one;
1-(3-((6,7-dichloro-2,2-dioxido-4,9-dihydro-[1,2,6]thiadiazino[4,3-g]indol-3 (1H)-yl)methyl) piperidin-1-yl)-2-(2-methoxyethoxy)ethan-1-one;
1-(3-((6,7-dichloro-2,2-dioxido-4,9-dihydro-[1,2,6]thiadiazino[4,3-g]indol-3 (1H)-yl)methyl) piperidin-1-yl)-5-methoxypentan-1-one;
tert-butyl(5-(4-((6,7-dichloro-2,2-dioxido-4,9-dihydro-[1,2,6]thiadiazino[4,3-g]indol-3 (1H)-yl)methyl) piperidin-1-yl)-5-oxopentyl) carbamate;
5-amino-1-(4-((6,7-dichloro-2,2-dioxido-4,9-dihydro-[1,2,6]thiadiazino[4,3-g]indol-3 (1H)-yl)methyl) piperidin-1-yl) pentan-1-one;
6,7-dichloro-N-methyl-3-(piperidin-3-ylmethyl)-1,3,4,9-tetrahydro-[1,2,6]thiadiazino[4,3-g]indole-8-carboxamide 2,2-dioxide;
tert-butyl 3-((6,7-dichloro-8-(methylcarbamoyl)-2,2-dioxido-4,9-dihydro-[1,2,6]thiadiazino[4,3-g]indol-3 (1H)-yl)methyl) piperidine-1-carboxylate;
3-((1-acetylpiperidin-3-yl)methyl)-6,7-dichloro-N-methyl-1,3,4,9-tetrahydro-[1,2,6]thiadiazino[4,3-g]indole-8-carboxamide 2,2-dioxide;
methyl 6,7-dichloro-3-(3-methoxypropyl)-1,3,4,9-tetrahydro-[1,2,6]thiadiazino[4,3-g]indole-8-carboxylate 2,2-dioxide;
ethyl 6,7-dichloro-3-(3-methoxypropyl)-1,3,4,9-tetrahydro-[1,2,6]thiadiazino[4,3-g]indole-8-carboxylate 2,2-dioxide;
6,7-dichloro-3-(3-methoxypropyl)-N-methyl-1,3,4,9-tetrahydro-[1,2,6]thiadiazino[4,3-g]indole-8-carboxamide 2,2-dioxide;
6,7-dichloro-3-((2-hydroxypyridin-4-yl)methyl)-N-methyl-1,3,4,9-tetrahydro-[1,2,6]thiadiazino[4,3-g]indole-8-carboxamide 2,2-dioxide;
4-(6,7-dichloro-2,2-dioxido-3-(piperidin-3-ylmethyl)-1,3,4,9-tetrahydro-[1,2,6]thiadiazino[4,3-g]indol-8-yl)butanamide;
4-(6,7-dichloro-3-(3-methoxypropyl)-2,2-dioxido-1,3,4,9-tetrahydro-[1,2,6]thiadiazino[4,3-g]indol-8-yl)butanoic acid;
4-(6,7-dichloro-3-(3-methoxypropyl)-2,2-dioxido-1,3,4,9-tetrahydro-[1,2,6]thiadiazino[4,3-g]indol-8-yl)butanamide;
4-(3-((1-acetylpiperidin-3-yl)methyl)-6,7-dichloro-2,2-dioxido-1,3,4,9-tetrahydro-[1,2,6]thiadiazino[4,3-g]indol-8-yl)butanamide;
4-(6,7-dichloro-3-((2-hydroxypyridin-4-yl)methyl)-2,2-dioxido-1,3,4,9-tetrahydro-[1,2,6]thiadiazino[4,3-g]indol-8-yl)butanamide;
tert-butyl 3-((8-(4-amino-4-oxobutyl)-6,7-dichloro-2,2-dioxido-4,9-dihydro-[1,2,6]thiadiazino[4,3-g]indol-3 (1H)-yl)methyl) piperidine-1-carboxylate;
4-(6,7-dichloro-2,2-dioxido-3-(piperidin-3-ylmethyl)-1,3,4,9-tetrahydro-[1,2,6]thiadiazino[4,3-g]indol-8-yl)butanoic acid;
methyl 4-(6,7-dichloro-3-((2-hydroxypyridin-4-yl)methyl)-2,2-dioxido-1,3,4,9-tetrahydro-[1,2,6]thiadiazino[4,3-g]indol-8-yl)butanoate;
ethyl 4-(6,7-dichloro-3-((2-hydroxypyridin-4-yl)methyl)-2,2-dioxido-1,3,4,9-tetrahydro-[1,2,6]thiadiazino[4,3-g]indol-8-yl)butanoate; and
4-(6,7-dichloro-3-((2-hydroxypyridin-4-yl)methyl)-2,2-dioxido-1,3,4,9-tetrahydro-[1,2,6]thiadiazino[4,3-g]indol-8-yl)butanoic acid.

18. A pharmaceutical composition comprising a compound of claim 1 or a pharmaceutically acceptable salt thereof, and a pharmaceutically acceptable diluent or carrier.

19. (canceled)

20. (canceled)

21. A method of treating cancer in a subject in need thereof,
comprising administering to said subject a therapeutically effective amount of a compound of claim 1 or a pharmaceutically acceptable salt thereof.

22. A method of modulating at least one activity selected from RBM39 activity and DCAF 15 activity, in a patient in need thereof, comprising administering to said patient a therapeutically effective amount of a compound of claim 1 or a pharmaceutically acceptable salt thereof.

23. The method according to claim 22, or a pharmaceutically acceptable salt thereof, wherein the at least one activity is RBM39.

24. The method according to claim 22, or a pharmaceutically acceptable salt thereof, wherein the at least one activity is DECAF 15 activity.
